(19) United States
(12) Patent Application Publication
(10) Pub. No.: US 2008/0293709 A1
(43) Pub. Date: Nov. 27, 2008

(54) Novel 2-Amino-Heterocycles Useful in the Treatment of Abeta-Related Pathologies

(75) Inventors: Jeffrey Albert, Wilmington, DE (US); Gianni Chessari, Cambridge (GB); Miles Stuart Congreve, Cambridge (GB); Phil Edwards, Wilmington, DE (US); Christopher Murray, Cambridge (GB); Sahil Patel, Cambridge (GB); Mark Sylvester, Wilmington, DE (US)

Correspondence Address: PEPPER HAMILTON LLP, 400 BERWYN PARK, 899 CASSATT ROAD, BERWYN, PA, 19312-1183, US

(73) Assignee: AstraZeneca AB, Sodertalje (SE)

(21) Appl. No.: 12/093,631

(22) PCT Filed: Nov. 13, 2006
(86) PCT No.: PCT/SE06/01283

§ 371 Date: May 30, 2008

Related U.S. Application Data
(60) Provisional application No. 60/737,327, filed on Nov. 15, 2005.

(51) Int. Cl. A61K 31/472; A61P 25/00; C07D 217/22; C07D 401/02; A61K 31/517; C07D 495/04; A61K 31/536; A61K 31/4725; C07D 239/72; C07D 265/14

(52) U.S. Cl. 514/230.5; 546/114; 546/143; 546/141; 544/283; 544/90; 514/310; 514/309; 514/266.4; 544/231

(57) ABSTRACT

This invention relates to novel compounds having the structural formula I below: and to their pharmaceutically acceptable salts, compositions and methods of use. These novel compounds provide a treatment or prophylaxis of cognitive impairment, Alzheimer Disease, neurodegeneration and dementia.

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products-amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases; this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994); This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO 05/097767, US2005/0282826).

The compounds of the present invention show improved properties compared to the potential inhibitors known in the art, e.g. improved hERG selectivity.

### DISCLOSURE OF THE INVENTION

Provided herein are novel compounds of structural formula I:

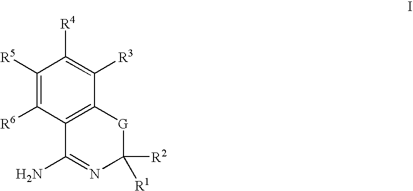

or a pharmaceutically acceptable salt, tautomer, or in vivo-hydrolysable precursor thereof, wherein:

G is O, NR⁷ or CR⁸R⁹;

R¹ is H, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 R¹⁴;

R² is Q or -L-Q;

or R¹ and R² together with the carbon atom to which they are attached form a 3-14 membered cycloalkyl group or 3-14 membered heterocycloalkyl group, each substituted by Cy² and optionally substituted by 1, 2, 3, 4 or 5 A⁴;

R³, R⁴, R⁵ and R⁶ are, independently, H, CN, NO₂, OR^{a}, SR^{a}, OC(O)R^{a}, OC(O)OR^{b}, OC(O)NR^{c}R^{d}, C(O)R^{a}, C(O)OR^{b}, C(O)NR^{c}R^{d}, NR^{c}R^{d}, NR^{c}C(O)R^{a}, NR^{c}C(O)OR^{b}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

R⁷ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 R¹⁴;

R⁸ and R⁹ are, independently, H, CN, NO₂, OR^{a}, SR^{a}, OC(O)R^{a}, OC(O)OR^{b}, C(O)OR^{b}, OC(O)NR^{c}R^{d}, NR^{c}R^{d}, NR^{c}C(O)R^{a}, NR^{c}C(O)OR^{b}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

or R⁸ and R⁹ together with the carbon atom to which they are attached form a 3-14 membered cycloalkyl or 3-14 membered heterocycloalkyl group, each optionally substituted by 1, 2 or 3 R¹⁴;

R¹² and R¹³ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};

R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};

Q is aryl, cycloalkyl, heteroaryl or heterocycloalkyl, each optionally substituted by 1, 2, 3, 4 or 5 Cy¹ or A¹;

L is C₂₋₁₀ alkenylenyl, C₂₋₁₀ alkynylenyl, (CR¹²R¹³)_{q}, (CR¹²R¹³)_{q1}O(CR¹²R¹³)_{q2}, (CR¹²R¹³)_{q1}S(CR¹²R¹³)_{q2}, (CR¹²R¹³)_{q1}SO₂(CR¹²R¹³)_{q2}, (CR¹²R¹³)_{q1}SO(CR¹²R¹³)_{q2}, (CR¹²R¹³)_{q1}CO(CR¹²R¹³)_{q2}, (CR¹²R¹³)_{q1}NR^{e}(CR¹²R¹³)_{q2}, or (CR¹²R¹³)_{q1}CONR^{e}(CR¹²R¹³)_{q2};

Cy¹ is aryl, heteroaryl, cycloalkyl, or heterocycloalkyl, each optionally substituted with 1, 2, 3, 4 or 5 A²;

Cy² is aryl, heteroaryl, cycloalkyl, or heterocycloalkyl, each optionally substituted with 1, 2, 3, 4 or 5 A³;

A¹ is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{e}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};

A², A³, and A⁴ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₁₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆alkyl, C₁₋₆haloalkyl, C₁₋₆haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} is H, C₁₋₄ alkyl, C₁₋₄ haloalkyl, C₂₋₄ alkenyl, C₂₋₄ alkynyl, or CO—(C₁₋₄ alkyl);

q is 1, 2, 3, 4, 5 or 6;

q1 is 0, 1, 2 or 3; and

q2 is 0, 1, 2 or 3;

with the provisos:

a) when G is NH or CH₂; R² is -L-Q; L is —CH₂, —CH═CH—, or —C≡C—; and R¹ is H or methyl, then Q is other than unsubstituted phenyl; and

b) when G is NR⁷ or CR⁸R⁹; R⁷ is H, methyl, or phenyl optionally substituted by halo; R⁸ and R⁹ are each, independently, H or methyl; R² is Q; and R¹ is H or methyl, then Q is aryl, cycloalkyl, heteroaryl, or heterocycloalkyl, each substituted by at least one Cy³ and optionally substituted by 1, 2 or 3 A⁴.

In some embodiments, R¹ is H, C₁₋₆ alkyl, C₁₋₆ haloalkyl, aryl, heteroaryl, arylalkyl, heteroarylalkyl, cycloalkylalkyl or heterocycloalkylalkyl, wherein the C₁₋₆ alkyl, C₁₋₆ haloalkyl, aryl, heteroaryl, arylalkyl, heteroarylalkyl, cycloalkylalkyl or heterocycloalkylalkyl is optionally substituted by 1, 2, 3, 4 or 5 R¹⁴.

In some embodiments, R¹ is H, C₁₋₆ alkyl, C₁₋₆ haloalkyl, aryl, heteroaryl, arylalkyl or heteroarylalkyl, wherein the C₁₋₆ alkyl, aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ is C₁₋₆ haloalkyl, aryl, heteroaryl, arylalkyl or heteroarylalkyl, wherein the aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R² is Q or -L-Q; and Q is aryl, cycloalkyl, heteroaryl, or heterocycloalkyl, each optionally substituted by 1, 2 or 3 A¹.

In some embodiments, R² is Q or -L-Q; and Q is aryl, cycloalkyl, heteroaryl, or heterocycloalkyl, each substituted by at least one Cy¹ and optionally substituted by 1, 2 or 3 A¹.

In some embodiments, R² is Q or -L-Q; and Q is aryl or heteroaryl, each substituted by at least one Cy¹ and optionally substituted by 1, 2 or 3 A¹.

In some embodiments, R² is Q or -L-Q; and Q is aryl substituted by at least one Cy¹ and optionally substituted by 1, 2 or 3 A¹.

In some embodiments, R¹ is Q or -L-Q; and Q is phenyl substituted by at least one Cy¹ and optionally substituted by 1, 2 or 3 A¹.

In some embodiments, R² is Q or -L-Q; and Q is phenyl substituted by Cy¹.

In some embodiments, R² is Q or -L-Q; Q is phenyl substituted by Cy¹; and Cy¹ is aryl or heteroaryl, each optionally substituted with 1, 2, 3, 4 of 5 A².

In some embodiments, R² is Q or -L-Q; Q is phenyl substituted by Cy¹; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R² is Q or -L-Q; Q is phenyl substituted by Cy¹, wherein the Cy¹ is substituted at the meta-position of the phenyl; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R² is Q.

In some embodiments, R² is -L-Q; and L is C₂₋₁₀ alkenylenyl, C₂₋₁₀ alkynylenyl or (CR¹²R¹³)_{q}.

In some embodiments, R² is -L-Q; and L is C₂₋₁₀ alkenylenyl, C₂₋₁₀ alkynylenyl or (CR¹²R¹³)_{q}.

In some embodiments, R² is -L-Q; and L is (CR¹²R¹³)_{q}.

In some embodiments, R² is -L-Q; L is (CR¹²R¹³)_{q}; and q is 2. In some embodiments, R¹ and R² together with the carbon atom to which they are attached form a 3-14 membered cycloalkyl group or 3-14 membered heterocycloalkyl group, each substituted by Cy² and optionally substituted by 1, 2 or 3 A⁴; and Cy² is aryl or heteroaryl, each optionally substituted with 1, 2, 3, 4 or 5 A³.

In some embodiments, R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; Cy² is aryl or heteroaryl, each optionally substituted with 1, 2 or 3 A³; and A³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; Cy² is phenyl substituted with 1 or 2 A³; and A³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³, R⁴, R⁵ and R⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀-alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴.

In some embodiments, R³, R⁴, R⁵ and R⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

In some embodiments, R³, R⁴, R⁵ and R⁶ are, independently, H.

In some embodiments, R⁴ is CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

In some embodiments, G is O.

In some embodiments, G is NR⁷ or CR⁸R⁹; and R⁷, R⁸ and R⁹ are each, independently, H, C₁₋₁₀ alkyl, C₁₋₁₀ haloalkyl, C₂₋₁₀ alkenyl, C₂₋₁₀ alkynyl, cycloalkyl, heterocycloalkyl, cycloalkylalkyl or heterocycloalkylalkyl.

In some embodiments, R¹ is C₁₋₆haloalkyl, aryl, heteroaryl, arylalkyl or heteroarylalkyl, wherein the aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; R² is Q; and Q is aryl or heteroaryl, each optionally substituted by 1, 2 or 3 A¹.

Also provided herein are novel compounds of structural formula II:

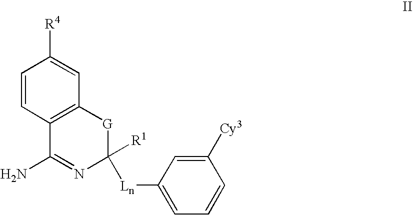

wherein:

R¹ is H, C₁₋₆ alkyl, C₁₋₆ haloalkyl, aryl, heteroaryl, arylalkyl or heteroarylalkyl, wherein the C₁₋₆ alkyl, aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

L is C₁₋₄ alkylenyl;

n is 0 or 1; and

Cy³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, L is CH₂CH₂; and Cy³ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Provided herein are novel compounds of structural formula IIIa or formula IIIb:

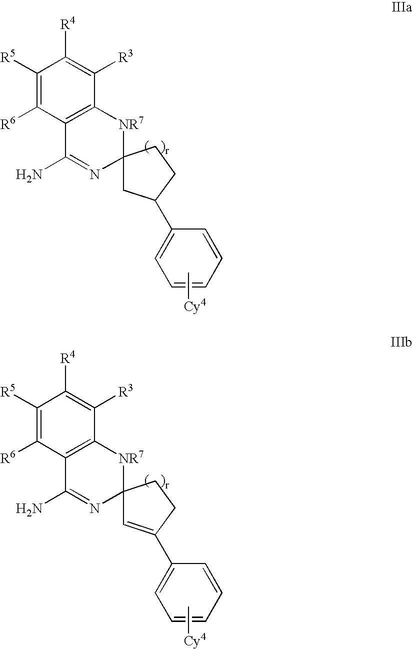

wherein:

r is 0, 1, 2 or 3; and

Cy⁴ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Provided herein are novel compounds of structural formula IVa or formula IVb:

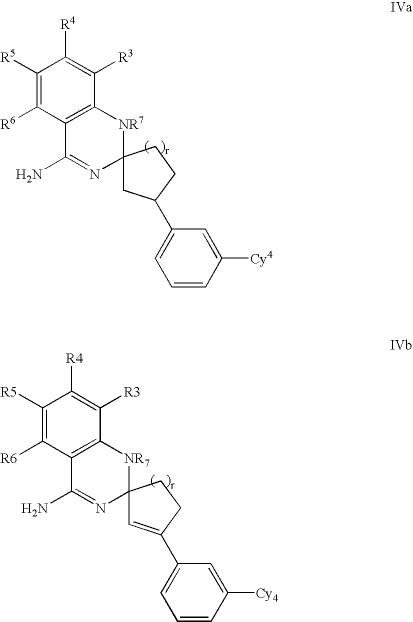

wherein:

r is 0, 1, 2 or 3; and

Cy⁴ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Also provided herein are novel compounds of structural formula V:

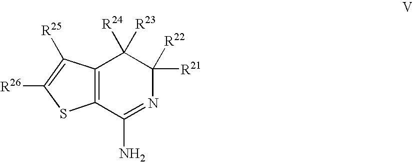

or a pharmaceutically acceptable salt, tautomer, or in vivo-hydrolysable precursor thereof, wherein:

R²¹ is H, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 R²⁹;

R²² is Q or -L-Q;

R², R²⁴, R²⁵ and R²⁶ are, independently, H, Si(C₁₋₁₀ alkyl)₃, CN, NO₂, OR^{a}, SR^{a}, OC(O)R^{a}, OC(O)OR^{b}, OC(O)NR^{c}R^{d}, C(O)R^{a}, C(O)OR^{b}, C(O)NR^{c}R^{d}, NR^{c}R^{d}, NR^{c}C(O)R^{a}, NR^{c}C(O)OR^{b}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R²⁹;

R²⁷ and R²⁸ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};

R²⁹ is halo, C₁₋₄ alkyl, C₁₋₄ haloalkyl, aryl, cycloalkyl, heteroaryl, heterocycloalkyl, CN, NO₂, OR^{a}, SR^{a′}, C(O)R^{b}, C(O)NR^{c′}R^{d′}, C(O)OR^{a′}, OC(O)R^{b}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};

Q is aryl, cycloalkyl, heteroaryl or heterocycloalkyl, each optionally substituted by 1, 2, 3, 4 or 5 Cy¹ or A¹;

L is C₂₋₁₀ alkenylenyl, C₂₋₁₀ alkynylenyl, (CR²⁷R²⁸)_{q}, (CR²⁷R²⁸)_{q1}O(CR²⁷R²⁸)_{q2}, (CR²⁷R²⁸)_{q1}S(CR²⁷R²⁸)_{q2}, (CR²⁷R²⁸)_{q1}SO₂(CR²⁷R²⁸)_{q2}, (C R²⁷R²⁸)_{q1}SO(C R²⁷R²⁸)_{q2}, (C R²⁷R²⁸)_{q1}CO(C R²⁷R²⁸)_{q2}, (C R²⁷R²⁸)^{q1}NR^{e}(C R²⁷R²⁸)_{q2}, or (C R²⁷R²⁸)_{q1}CONR^{e}(C R²⁷R²⁸)_{q2};

Cy¹ is aryl, heteroaryl, cycloalkyl, or heterocycloalkyl, each optionally substituted with 1, 2, 3, 4 or 5 A²;

A¹ is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{d}, NR^{c}S(O)R^{d}, NR^{c}S(O)₂R^{b}, S(O)R^{b}, S(O)NR^{c}R^{d}, S(O)₂R^{b}, or S(O)₂NR^{c}R^{d};

A² is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{e}, S(O)₂NR^{c}R^{d}, C₁₋₄alkoxy, C₁₋₄ haloalkoxy, amino, C₁₋₄alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂ or S(O)₂NR^{c}R^{d};

R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl; C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} is H, C₁₋₄ alkyl, C₁₋₄ haloalkyl, C₂₋₄ alkenyl, C₂₋₄ alkynyl, or CO—(C₁₋₄ alkyl);

q is 1, 2, 3, 4, 5 or 6;

q1 is 0, 1, 2 or 3; and

q2 is 0.1, 2 or 3;

with the provisos:

when R²¹, R²³ and R²⁴ are each H, and R²² is Q, then Q is aryl, cycloalkyl, heteroaryl, or heterocycloalkyl, each substituted by at least one Cy¹ and optionally substituted by 1, 2 or 3 A¹; and

when R²¹, R²² and R²³ are each H, R²² is -L-Q and L is —C≡C—, then Q is other than unsubstituted phenyl.

In some embodiments, R²¹ is H, C₁₋₆ alkyl, C₁₋₆ haloalkyl, aryl, heteroaryl, arylalkyl, heteroarylalkyl, cycloalkylalkyl or heterocycloalkylalkyl, wherein the C₁₋₆ alkyl, C₁₋₆ haloalkyl, aryl, heteroaryl, arylalkyl, heteroarylalkyl, cycloalkylalkyl or heterocycloalkylalkyl is optionally substituted by 1, 2, 3, 4 or 5 R²⁹.

In some embodiments, R²¹ is H, C₁₋₆ alkyl, C₁₋₆ haloalkyl, arylalkyl, heteroarylalkyl, cycloalkylalkyl or heterocycloalkylalkyl, wherein each of the C₁₋₆ alkyl, C₁₋₆ haloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²¹ is C₁₋₆ alkyl or C₁₋₆ haloalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²¹ is C₁₋₆ alkyl or C₁₋₆ haloalkyl.

In some embodiments, R²¹ is C₁₋₆haloalkyl.

In some embodiments, R²¹ is trifluoromethyl.

In some embodiments, R²¹ is H.

In some embodiments, R²² is Q or -L-Q; and Q is aryl, cycloalkyl, heteroaryl, or heterocycloalkyl, each optionally substituted by 1, 2 or 3 A¹.

In some embodiments, R²² is Q or -L-Q; and Q is aryl, cycloalkyl, heteroaryl, or heterocycloalkyl, each substituted by at least one Cy¹ and optionally substituted by 1, 2 or 3 A¹.

In some embodiments, R²² is Q or -L-Q; and Q is aryl or heteroaryl, each substituted by at least one Cy¹ and optionally substituted by 1, 2 or 3 A¹.

In some embodiments, R²² is Q or -L-Q; and Q is aryl substituted by at least one Cy¹ and optionally substituted by 1, 2 or 3 A¹.

In some embodiments, R²² is Q or -L-Q; and Q is phenyl substituted by at least one Cy¹ and optionally substituted by 1, 2 or 3 A¹.

In some embodiments, R²² is Q or -L-Q; and Q is phenyl substituted by Cy¹.

In some embodiments, R²² is Q or -L-Q; Q is phenyl substituted by Cy¹; and Cy¹ is aryl or heteroaryl, each optionally substituted with 1, 2, 3, 4 or 5 A².

In some embodiments, R²² is Q or -L-Q; Q is phenyl substituted by Cy¹; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²² is Q or -L-Q; Q is phenyl substituted by Cy¹, wherein the Cy¹ is substituted at the meta-position of the phenyl; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²² is Q.

In some embodiments, R²² is -L-Q; and L is C₂₋₁₀ alkenylenyl or (C R²⁷R²⁸)_{q}.

In some embodiments, R²² is -L-Q; and L is (C R²⁷R²⁸)_{q}.

In some embodiments, R²³, R²⁴, R²⁵ and R²⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R²⁹.

In some embodiments, R²³, R²⁴, R²⁵ and R²⁶ are, independently, H, Si(C₁₋₁₀ alkyl)₃, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

In some embodiments, R²³, R²⁴, R²⁵ and R²⁶ are, independently, H, Si(C₁₋₁₀ alkyl)₃, CN, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, OH, C₁₋₄ alkoxy, C₁₋₄ alkyl, C₁₋₄ haloalkyl, aryl, cycloalkyl, heteroaryl and heterocycloalkyl.

In some embodiments, R²³ and R²⁴ are, independently, H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

In some embodiments, R²³ and R²⁴ are, independently, H or C₁₋₁₀ alkyl.

In some embodiments, R²⁵ and R²⁶ are, independently, H, Si(C₁₋₁₀ alkyl)₃, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

Also provided herein are novel compounds of structural formula VI:

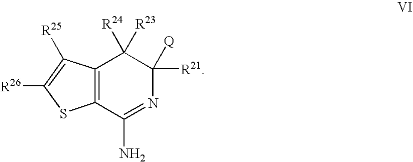

In some embodiments, R²¹ is H, C₁₋₆ alkyl or C₁₋₆ haloalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²¹ is C₁₋₆ alkyl or C₁₋₆ haloalkyl.

In some embodiments, R²¹ is C₁₋₆ haloalkyl.

In some embodiments, Q is aryl, cycloalkyl, heteroaryl or heterocycloalkyl, each substituted by at least one Cy¹ and optionally substituted by 1, 2 or 3 A¹.

In some embodiments, Q is aryl substituted by at least one Cy¹ and optionally substituted by 1, 2 or 3 A¹.

In some embodiments, Q is phenyl substituted by at least one Cy¹ and optionally substituted by 1, 2 or 3 A¹.

In some embodiments, Q is phenyl substituted by at least one Cy¹ at the meta-position and optionally substituted by 1, 2 or 3 A¹.

In some embodiments, R²¹ is H, C₁₋₆ alkyl or C₁₋₆ haloalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²¹ is H, C₁₋₆ alkyl or C₁₋₆ haloalkyl.

In some embodiments, R²¹ is H.

In some embodiments, R²³ and R²⁴ are, independently, H or C₁₋₁₀ alkyl.

The present invention further provides compositions comprising a compound of any of the formulas described herein, or a pharmaceutically acceptable salt, tautomer or in vivo-hydrolysable precursor thereof, and at least one pharmaceutically acceptable carrier, diluent or excipient.

The present invention further provides methods of modulating activity of BACE comprising contacting the BACE with a compound of any of the formulas described herein, or a pharmaceutically acceptable salt, tautomer or in vivo-hydrolysable precursor thereof.

The present invention further provides methods of treating or preventing an Aβ-related pathology in a patient, comprising administering to the patient a therapeutically effective amount of a compound of any of the formulas described herein, or a pharmaceutically acceptable salt, tautomer or in vivo-hydrolysable precursor thereof.

The present invention further provides a compound of any of the formulas described herein, or a pharmaceutically acceptable salt, tautomer or in vivo-hydrolysable precursor thereof, described herein for use as a medicament.

The present invention further provides a compound of any of the formulas described herein, or a pharmaceutically acceptable salt, tautomer or in vivo-hydrolysable precursor thereof, described herein for the manufacture of a medicament.

### DETAILED DESCRIPTION OF THE INVENTION

Provided herein are novel compounds of structural formula I:

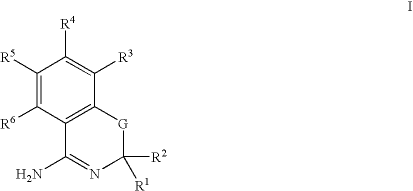

or a pharmaceutically acceptable salt, tautomer, or in vivo-hydrolysable precursor thereof.

In some embodiments, G is O, NR⁷ or CR⁸R⁹, or any subgroup thereof. In some embodiments, G is O. In some embodiments, G is NR⁷ or CR⁸R⁹.

In some embodiments, R¹ is H, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 R¹⁴, or any subgroup thereof. In some embodiments, R¹ is H, C₁₋₆ alkyl, C₁₋₆ haloalkyl, aryl, heteroaryl, arylalkyl, heteroarylalkyl, cycloalkylalkyl or heterocycloalkylalkyl, wherein the C₁₋₆ alkyl, C₁₋₆ haloalkyl, aryl, heteroaryl, arylalkyl, heteroarylalkyl, cycloalkylalkyl or heterocycloalkylalkyl is optionally substituted by 1, 2, 3, 4 or 5 R¹⁴. In some embodiments, R¹ is H, C₁₋₆ alkyl, C₁₋₆ haloalkyl, aryl, heteroaryl, arylalkyl or heteroarylalkyl, wherein the C₁₋₆ alkyl, aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, R¹ is C₁₋₆haloalkyl, aryl, heteroaryl, arylalkyl or heteroarylalkyl, wherein the aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R² is Q or -L-Q. In some embodiments, R² is Q. In some embodiments, R² is -L-Q.

In some embodiments, R¹ and R² together with the carbon atom to which they are attached form a 3-14 membered cycloalkyl group or 3-14 membered heterocycloalkyl group, each substituted by Cy² and optionally substituted by 1, 2, 3, 4 or 5 A⁴, or any subgroup thereof. In some embodiments, R¹ and R² together with the carbon atom to which they are attached form a 3-14 membered cycloalkyl group or 3-14 membered heterocycloalkyl group, each substituted by Cy² and optionally substituted by 1, 2 or 3 A⁴. In some embodiments, R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R³, R⁴, R⁵ and R⁶ are, independently, H, CN, NO₂, OR^{a}, SR^{a}, OC(O)R^{a}, OC(O)OR^{b}, OC(O)NR^{c}R^{d}, C(O)R^{a}, C(O)OR^{b}, C(O)NR^{c}R^{d}, NR^{c}R^{d}, NR^{c}C(O)R^{a}, NR^{c}C(O)OR^{b}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴, or any subgroup thereof. In some embodiments, R³, R⁴, R⁵ and R⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴. In some embodiments, R³, R⁴, R⁵ and R⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

In some embodiments, R³, R⁴, R⁵ and R⁶ are, independently, H.

In some embodiments, R⁷ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 R¹⁴, or any subgroup thereof. In some embodiments, R⁷ is H, C₁₋₁₀ alkyl, C₁₋₁₀ haloalkyl, C₂₋₁₀ alkenyl, C₂₋₁₀ alkynyl, cycloalkyl, heterocycloalkyl, cycloalkylalkyl or heterocycloalkylalkyl.

In some embodiments, R⁸ and R⁹ are, independently, H, CN, NO₂, OR^{a}, SR^{a}, OC(O)R^{a}, OC(O)OR^{b}, C(O)OR^{b}, OC(O)NR^{c}R^{d}, NR^{c}R^{d}, NR^{c}C(O)R^{a}, NR^{c}C(O)OR^{b}, NR^{e}S(O)₂R^{b}, S(O)R^{a}, S(O)NR^{c}R^{d}, S(O)₂R^{a}, S(O)₂NR^{e}R^{d}, C₁₋₁₀alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴, or any subgroup thereof.

In some embodiments, R⁵ and R⁹ together with the carbon atom to which they are attached form a 3-14 membered cycloalkyl or 3-14 membered heterocycloalkyl group, each optionally substituted by 1, 2 or 3 R¹⁴.

In some embodiments, R¹² and R¹³ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof.

In some embodiments, R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof.

In some embodiments, Q is aryl, cycloalkyl, heteroaryl or heterocycloalkyl, each optionally substituted by 1, 2, 3, 4 or 5 Cy¹ or A¹, or any subgroup thereof. In some embodiments, Q is aryl, cycloalkyl, heteroaryl, or heterocycloalkyl, each optionally substituted by 1, 2 or 3 A¹. In some embodiments, Q is aryl, cycloalkyl, heteroaryl, or heterocycloalkyl, each substituted by at least one Cy¹ and optionally substituted by 1, 2 or 3 A¹. In some embodiments, Q is aryl or heteroaryl, each substituted by at least one Cy¹ and optionally substituted by 1, 2 or 3 A¹. In some embodiments, Q is aryl substituted by at least one Cy¹ and optionally substituted by 1, 2 or 3 A¹. In some embodiments, Q is phenyl substituted by at least one Cy¹ and optionally substituted by 1, 2 or 3 A¹. In some embodiments, Q is phenyl substituted by Cy¹. In some embodiments, Q is phenyl substituted by Cy¹, wherein the Cy¹ is substituted at the meta-position of the phenyl. In some embodiments, Q is aryl or heteroaryl, each optionally substituted by 1, 2 or 3 A¹.

In some embodiments, L is C₂₋₁₀ alkenylenyl, C₂₋₁₀ alkynylenyl, (CR¹²R¹³)_{q}, (CR¹²R¹³)_{q1}O(CR¹²R¹³)_{q2}, (CR¹²R¹³)_{q1}S(CR¹²R¹³)_{q2}, (CR¹²R¹³)_{q1}SO₂(CR¹²R¹³)_{q2}, (CR¹²R¹³)_{q1}SO(CR¹²R¹³)_{q2}, (CR¹²R¹³)_{q1}CO(CR¹²R¹³)_{q2}, (CR¹²R¹³)_{q1}NR^{e}(CR¹²R¹³)_{q2}, or (CR¹²R¹³)_{q1}CONR^{e}(CR¹²R¹³)_{q2}, or any subgroup thereof. In some embodiments, L is C₂₋₁₀ alkenylenyl, C₂₋₁₀ alkynylenyl or (CR¹²R¹³)_{q}. In some embodiments, L is (CR¹²R¹³)_{q}.

In some embodiments, Cy¹ is aryl, heteroaryl, cycloalkyl, or heterocycloalkyl, or any subgroup thereof, each optionally substituted with 1, 2, 3, 4 or 5 A², or any subgroup thereof. In some embodiments, Cy¹ is aryl or heteroaryl, each optionally substituted with 1, 2, 3, 4 or 5 A². In some embodiments, Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, Cy² is aryl, heteroaryl, cycloalkyl, or heterocycloalkyl, or any subgroup thereof, each optionally substituted with 1, 2, 3, 4 or 5 A³, or any subgroup thereof. In some embodiments, Cy² is aryl or heteroaryl, each optionally substituted with 1, 2, 3, 4 or 5 A³. In some embodiments, Cy² is aryl or heteroaryl, each optionally substituted with 1, 2 or 3 A³. In some embodiments, Cy² is phenyl substituted with 1 or 2 A³.

In some embodiments, A¹ is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{e}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{e}, or any subgroup thereof.

In some embodiments, A², A³, and A⁴ are each, independently, halo, CN, NO₂, OR^{a}, SR^{a}, C(O)R^{b}, C(O)NR^{c}R^{d}C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}OC(O)R^{b}, OC(O)NR^{c}R^{d}, NR^{c}R^{d}, NR^{c}C(O)R^{d}NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof. In some embodiments, A³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl, or any subgroup thereof.

In some embodiments, R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} together with the N atom to which they are attached form a 4-, 5-, 6- or 7-membered heterocycloalkyl group.

In some embodiments, R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c′} and R^{d′} together with the N atom to which they are attached form a 4-, 5-, 6- or 7-membered heterocycloalkyl group, or any subgroup thereof.

In some embodiments, R^{e} is H, C₁₋₄ alkyl, C₁₋₄ haloalkyl, C₂₋₄ alkenyl, C₂₋₄ alkynyl, or CO—(C₁₋₄ alkyl), or any subgroup thereof.

In some embodiments, q is 1, 2, 3, 4, 5 or 6, or any subgroup thereof. In some embodiments, q is 2.

In some embodiments, q1 is 0, 1, 2 or 3, or any subgroup thereof.

In some embodiments, q2 is 0, 1, 2 or 3, or any subgroup thereof.

When G is NH or CH₂, R² is -L-Q, L is —CH₂, —CH═CH—, or —C≡C—, and R¹ is H or methyl, however, then Q is other than unsubstituted phenyl.

When G is NR⁷ or CR⁸R⁹, R⁷ is H, methyl, or phenyl optionally substituted by halo, R⁸ and R⁹ are each, independently, H or methyl, R² is Q, and R¹ is H or methyl, however, then Q is aryl, cycloalkyl, heteroaryl, or heterocycloalkyl, each substituted by at least one Cy³ and optionally substituted by 1, 2 or 3 A⁴.

In some embodiments, R¹ is Q or -L-Q; and Q is aryl, cycloalkyl, heteroaryl, or heterocycloalkyl, each optionally substituted by 1, 2 or 3 A¹.

In some embodiments, R² is Q or -L-Q; and Q is aryl, cycloalkyl, heteroaryl, or heterocycloalkyl, each substituted by at least one Cy¹ and optionally substituted by 1, 2 or 3 A¹.

In some embodiments, R² is Q or -L-Q; and Q is aryl or heteroaryl, each substituted by at least one Cy¹ and optionally substituted by 1, 2 or 3 A¹.

In some embodiments, R² is Q or -L-Q; and Q is aryl substituted by at least one Cy¹ and optionally substituted by 1, 2 or 3 A¹.

In some embodiments, R² is Q or -L-Q; and Q is phenyl substituted by at least one Cy¹ and optionally substituted by 1, 2 or 3 A¹.

In some embodiments, R² is Q or -L-Q; and Q is phenyl substituted by Cy¹.

In some embodiments, R² is Q or -L-Q; Q is phenyl substituted by Cy¹; and Cy¹ is aryl or heteroaryl, each optionally substituted with 1, 2, 3, 4 or 5 A².

In some embodiments, R² is Q or -L-Q; Q is phenyl substituted by Cy¹; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ is Q or -L-Q; Q is phenyl substituted by Cy¹, wherein the Cy¹ is substituted at the meta-position of the phenyl; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ is -L-Q; and L is C₂₋₁₀ alkenylenyl, C₂₋₁₀ alkynylenyl or (CR¹²R¹³)_{q}.

In some embodiments, R² is -L-Q; and L is C₂₋₁₀-alkenylenyl, C₂₋₁₀ alkynylenyl or (CR¹²R¹³)_{q}.

In some embodiments, R² is -L-Q; and L is (CR¹²R¹³)_{q}.

In some embodiments, R² is -L-Q; L is (CR¹²R¹³)_{q}; and q is 2.

In some embodiments, R¹ and R² together with the carbon atom to which they are attached form a 3-14 membered cycloalkyl group or 3-14 membered heterocycloalkyl group, each substituted by Cy² and optionally substituted by 1, 2 or 3 A⁴; and Cy² is aryl or heteroaryl, each optionally substituted with 1, 2, 3, 4 or 5 A³.

In some embodiments, R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; Cy² is aryl or heteroaryl, each optionally substituted with 1, 2 or 3 A³; and A³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; Cy² is phenyl substituted with 1 or 2 A³; and A³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R⁴ is CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀-alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

In some embodiments, G is NR⁷ or CR⁸R⁹; and R⁷, R⁸ and R⁹ are each, independently, H, C₁₋₁₀ alkyl, C₁₋₁₀ haloalkyl, C₂₋₁₀ alkenyl, C₂₋₁₀ alkynyl, cycloalkyl, heterocycloalkyl, cycloalkylalkyl or heterocycloalkylalkyl.

In some embodiments, R¹ is C₁₋₆ haloalkyl, aryl, heteroaryl, arylalkyl or heteroarylalkyl, wherein the aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl; R² is Q; and Q is aryl or heteroaryl, each optionally substituted by 1, 2 or 3 A¹.

Also provided herein are novel compounds of structural formula II:

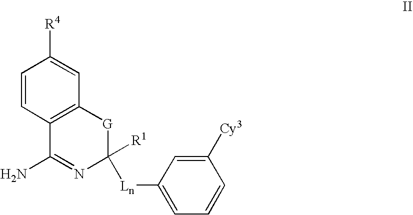

or a pharmaceutically acceptable salt, tautomer, or in vivo-hydrolysable precursor thereof.

In some embodiments, R¹ is H, C₁₋₆ alkyl, C₁₋₆ haloalkyl, aryl, heteroaryl, arylalkyl or heteroarylalkyl, or any subgroup thereof, wherein the C₁₋₆ alkyl, aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

In some embodiments, L is C₁₋₄ alkylenyl. In some embodiments, L is CH₂CH₂.

In some embodiments, n is 0 or 1.

In some embodiments, Cy³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. In some embodiments, Cy³ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, L is CH₂CH₂; and Cy³ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

Provided herein are novel compounds of structural formula IIIa or formula IIIb:

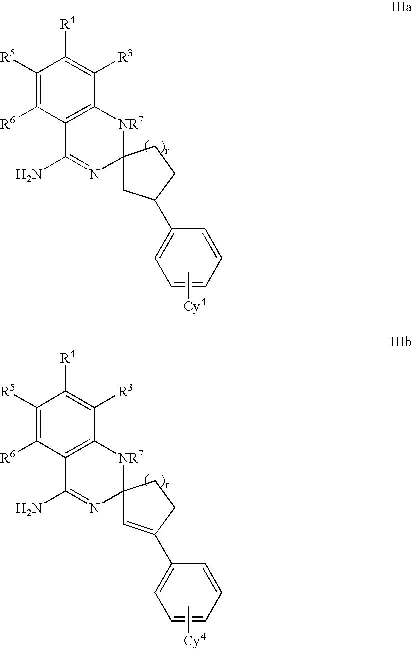

or a pharmaceutically acceptable salt, tautomer, or in vivo-hydrolysable precursor thereof.

In some embodiments, r is 0, 1, 2 or 3.

In some embodiments, Cy⁴ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

Provided herein are novel compounds of structural formula IVa or formula IVb:

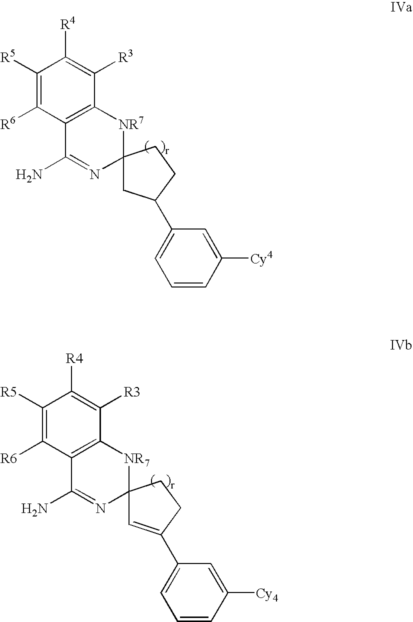

or a pharmaceutically acceptable salt, tautomer, or in vivo-hydrolysable precursor thereof.

In some embodiments, r is 0, 1, 2 or 3.

In some embodiments, Cy⁴ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof.

Also provided herein are novel compounds of structural formula V:

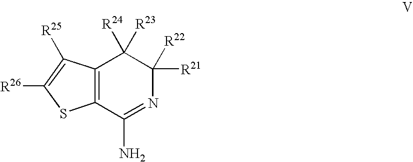

or a pharmaceutically acceptable salt, tautomer, or in vivo-hydrolysable precursor thereof.

In some embodiments, R²¹ is H, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 R²⁹, or any subgroup thereof. In some embodiments, R²¹ is H, C₁₋₆ alkyl, C₁₋₆ haloalkyl, aryl, heteroaryl, arylalkyl, heteroarylalkyl, cycloalkylalkyl or heterocycloalkylalkyl, wherein the C₁₋₆ alkyl, C₁₋₆ haloalkyl, aryl, heteroaryl, arylalkyl, heteroarylalkyl, cycloalkylalkyl or heterocycloalkylalkyl is optionally substituted by 1, 2, 3, 4 or 5 R²⁹. In some embodiments, R²¹ is H, C₁₋₆ alkyl, C₁₋₆ haloalkyl, arylalkyl, heteroarylalkyl, cycloalkylalkyl or heterocycloalkylalkyl, wherein each of the C₁₋₆ alkyl, C₁₋₆ haloalkyl, arylalkyl, heteroarylalkyl, cycloalkylalkyl or heterocycloalkylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, R²¹ is C₁₋₆ alkyl or C₁₋₆ haloalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl. In some embodiments, R²¹ is C₁₋₆ alkyl or C₁₋₆ haloalkyl. In some embodiments, R²¹ is C₁₋₆haloalkyl. In some embodiments, R²¹ is trifluoromethyl. In some embodiments, R²¹ is H.

In some embodiments, R²² is Q or -L-Q. In some embodiments, R²² is Q. In some embodiments, R²² is -L-Q.

In some embodiments, R²³, R²⁴, R²⁵ and R²⁶ are, independently, H, Si(C₁₋₁₀ alkyl)₃, CN, NO₂, OR^{a}, SR^{a}, OC(O)R^{a}, OC(O)OR^{b}, OC(O)NR^{c}R^{d}, C(O)R^{a}, C(O)OR^{b}, C(O)NR^{c}R^{d}, NR^{c}R^{d}, NR^{c}C(O)R^{a}, NR^{c}C(O)OR^{b}, NR^{e}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₁₀ alkyl, C₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R²⁹, or any subgroup thereof. In some embodiments, R²³, R²⁴, R²⁵ and R²⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R²⁹. In some embodiments, R²³, R²⁴, R²⁵ and R²⁶ are, independently, H, Si(C₁₋₁₀ alkyl)₃, CN, C(O)R^{a}, C(O)OR^{b}, C(O)NR^{c}R^{d}, C₁₋₁₀alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{a′}, NR^{c′}C(O)OR^{a′} and NR^{c′}S(O)₂R^{b′}. In some embodiments, R²³, R²⁴, R²⁵ and R²⁶ are, independently, H, Si(C₁₋₁₀ alkyl)₃, CN, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, OH, C₁₋₄ alkoxy, C₁₋₄ alkyl, C₁₋₄ haloalkyl, aryl, cycloalkyl, heteroaryl and heterocycloalkyl. In some embodiments, R²³ and R²⁴ are, independently, H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl. In some embodiments, R²³ and R²⁴ are, independently, H or C₁₋₁₀ alkyl. In some embodiments, R²⁵ and R²⁶ are, independently, H, Si(C₁₋₁₀ alkyl)₃, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

In some embodiments, R²⁷ and R²⁸ are each, independently, H, halo, C₁₋₄ alkyl, C₁₋₄ haloalkyl, aryl, cycloalkyl, heteroaryl, heterocycloalkyl, CN, NO₂, OR^{a}, SR^{a}, C(O)R^{b}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof.

In some embodiments, R²⁹ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′}, or any subgroup thereof.

In some embodiments, Q is aryl, cycloalkyl, heteroaryl or heterocycloalkyl, or any subgroup thereof, each optionally substituted by 1, 2, 3, 4 or 5 Cy¹ or A¹. In some embodiments, Q is aryl, cycloalkyl, heteroaryl, or heterocycloalkyl, each optionally substituted by 1, 2 or 3 A¹. In some embodiments, Q is aryl, cycloalkyl, heteroaryl, or heterocycloalkyl, each substituted by at least one Cy¹ and optionally substituted by 1, 2 or 3 A¹. In some embodiments, Q is aryl or heteroaryl, each substituted by at least one Cy¹ and optionally substituted by 1, 2 or 3 A¹. In some embodiments, Q is aryl substituted by at least one Cy¹ and optionally substituted by 1, 2 or 3 A¹. In some embodiments, Q is phenyl substituted by at least one Cy¹ and optionally substituted by 1, 2 or 3 A¹. In some embodiments, Q is phenyl substituted by Cy¹. In some embodiments, Q is phenyl substituted by Cy¹. In some embodiments, Q is phenyl substituted by Cy¹. In some embodiments, Q is phenyl substituted by Cy¹; wherein the Cy¹ is substituted at the meta-position of the phenyl

In some embodiments, L is C₂₋₁₀ alkenylenyl, C₂₋₁₀ alkynylenyl, (CR²⁷R²⁸)_{q}, (CR²⁷R²⁸)_{q1}O(CR²⁷R²⁸)_{q2}, (CR²⁷R²⁸)_{q1}S(CR²⁷R²⁸)_{q2}, (CR²⁷R²⁸)_{q1}SO₂(CR²⁷R²⁸)_{q2}, (CR²⁷R²⁸)_{q1}SO(CR¹⁷R²⁸)_{q2}, (CR²⁷R²⁸)_{q1}CO(CR²⁷R²⁸)_{q2}, (CR²⁷R²⁸)_{q1}NR^{e}(CR²⁷R²⁸)_{q2}, or (CR²⁷R²⁸)_{q1}CONR^{e}(CR²⁷R²⁸)_{q2}, or any subgroup thereof. In some embodiments, L is C₂₋₁₀ alkenylenyl or (CR²⁷R²⁸)_{q}. In some embodiments, L is (CR²⁷R²⁸)_{q}.

In some embodiments, Cy¹ is aryl, heteroaryl, cycloalkyl, or heterocycloalkyl, or any subgroup thereof, each optionally substituted with 1, 2, 3, 4 or 5 A², or any subgroup thereof. In some embodiments, Cy¹ is aryl or heteroaryl, each optionally substituted with 1, 2, 3, 4 or 5 A². In some embodiments, Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, A¹ is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{e}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl or heterocycloalkylalkyl, or any subgroup thereof, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof.

In some embodiments, A² is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄alkoxy, C₁₋₄haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, or any subgroup thereof, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d}, or any subgroup thereof.

In some embodiments, R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl, or any subgroup thereof.

In some embodiments, R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, or any subgroup thereof, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c} and R^{d} together with the N atom to which they are attached form a 4-, 5-, 6- or 7-membered heterocycloalkyl group, or any subgroup thereof.

In some embodiments, R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, or any subgroup thereof, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl, or any subgroup thereof.

In some embodiments, R^{c′} and R^{d′} together with the N atom to which they are attached form a 4-, 5-, 6- or 7-membered heterocycloalkyl group, or any subgroup thereof.

In some embodiments, R^{e} is H, C₁₋₄ alkyl, C₁₋₄ haloalkyl, C₂₋₄ alkenyl, C₂₋₄ alkynyl, or CO—(C₁₋₄ alkyl), or any subgroup thereof.

In some embodiments, q is 1, 2, 3, 4, 5 or 6, or any subgroup thereof.

In some embodiments, q1 is 0, 1, 2 or 3, or any subgroup thereof.

In some embodiments, q2 is 0, 1, 2 or 3, or any subgroup thereof.

When R²¹, R²³ and R²⁴ are each H, and R²² is Q, however, then Q is aryl, cycloalkyl, heteroaryl, or heterocycloalkyl, each substituted by at least one Cy¹ and optionally substituted by 1, 2 or 3 A¹.

When R²¹, R²³ and R²⁴ are each H; R²² is -L-Q and L is —C≡C—, however, then Q is other than unsubstituted phenyl.

In some embodiments, R²² is Q or -L-Q; and Q is aryl, cycloalkyl, heteroaryl, or heterocycloalkyl, each optionally substituted by 1, 2 or 3 A¹.

In some embodiments, R²² is Q or -L-Q; and Q is aryl, cycloalkyl, heteroaryl, or heterocycloalkyl, each substituted by at least one Cy¹ and optionally substituted by 1, 2 or 3 A¹.

In some embodiments, R²² is Q or -L-Q; and Q is aryl or heteroaryl, each substituted by at least one Cy¹ and optionally substituted by 1, 2 or 3 A¹.

In some embodiments, R²² is Q or -L-Q; and Q is aryl substituted by at least one Cy¹ and optionally substituted by 1, 2 or 3 A¹.

In some embodiments, R²² is Q or -L-Q; and Q is phenyl substituted by at least one Cy¹ and optionally substituted by 1, 2 or 3 A¹.

In some embodiments, R²² is Q or -L-Q; and Q is phenyl substituted by Cy¹.

In some embodiments, R²² is Q or -L-Q; Q is phenyl substituted by Cy¹; and Cy¹ is aryl or heteroaryl, each optionally substituted with 1, 2, 3, 4 or 5 A².

In some embodiments, R²² is Q or -L-Q; Q is phenyl substituted by Cy¹; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²² is Q or -L-Q; Q is phenyl substituted by Cy¹, wherein the Cy¹ is, substituted at the meta-position of the phenyl; and Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

In some embodiments, R²² is Q.

In some embodiments, R²² is -L-Q; and L is C₂₋₁₀ alkenylenyl or (CR²⁷R²⁸)_{q}.

In some embodiments, R²² is -L-Q; and L is (CR²⁷R²⁸)_{q}.

In some embodiments, R²³, R²⁴, R²⁵ and R²⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R²⁹.

In some embodiments, R²³, R²⁴, R²⁵ and R²⁶ are, independently, H, Si(C₁₋₁₀ alkyl)₃, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

In some embodiments, R²³, R²⁴, R²⁵ and R²⁶ are, independently, H, Si(C₁₋₁₀ alkyl)₃, CN, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, OH, C₁₋₄ alkoxy, C₁₋₄ alkyl, C₁₋₄ haloalkyl, aryl, cycloalkyl, heteroaryl and heterocycloalkyl.

In some embodiments, R²³ and R²⁴ are, independently, H, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

In some embodiments, R²³ and R²⁴ are, independently, H or C₁₋₁₀ alkyl.

In some embodiments, R²⁵ and R²⁶ are, independently, H, Si(C₁₋₁₀ alkyl)₃, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl.

Also provided herein are novel compounds of structural formula VI:

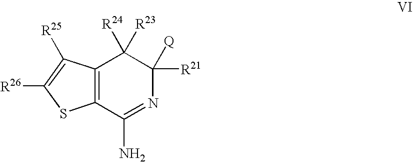

or a pharmaceutically acceptable salt, tautomer, or in vivo-hydrolysable precursor thereof.

In some embodiments, R²¹ is H, C₁₋₆ alkyl or C₁₋₆ haloalkyl, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. Other variables are as described above. In some embodiments, R²¹ is C₁₋₆ alkyl or C₁₋₆ haloalkyl. In some embodiments, R²¹ is C₁₋₆ haloalkyl. In some embodiments, R²¹ is H, C₁₋₆ alkyl or C₁₋₆ haloalkyl, or any subgroup thereof, each optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl, or any subgroup thereof. In some embodiments, R²¹ is H, C₁₋₆ alkyl or C₁₋₆ haloalkyl, or any subgroup thereof. In some embodiments, R²¹ is H.

In some embodiments, Q is aryl, cycloalkyl, heteroaryl or heterocycloalkyl, or any subgroup thereof, each substituted by at least one Cy¹ and optionally substituted by 1, 2 or 3 A¹. In some embodiments, Q is aryl substituted by at least one Cy¹ and optionally substituted by 1, 2 or 3 A¹. In some embodiments, Q is phenyl substituted by at least one Cy¹ and optionally substituted by 1, 2 or 3 A¹. In some embodiments, Q is phenyl substituted by at least one Cy¹ at the meta-position and optionally substituted by 1, 2 or 3 A¹.

In some embodiments, R²³ and R¹⁴ are, independently, H or C₁₋₁₀ alkyl.

Compounds of the invention include, for example:
3-(3′-Methoxybiphenyl-3-yl)-3,4-dihydroisoquinolin-1-amine trifluoroacetate;
3-(3-Bromophenyl)-3,4-dihydroisoquinolin-1-amine trifluoroacetate;
3-Biphenyl-3-yl-3,4-dihydroisoquinolin-1-amine trifluoroacetate;
3-Phenyl-3-(trifluoromethyl)-3,4-dihydroisoquinolin-1-amine trifluoroacetate;
3-(3′-Methoxybiphenyl-3-yl)-3-(trifluoromethyl)-3,4-dihydroisoquinolin-1-amine trifluoroacetate;
3-(3-Bromophenyl)-3-(trifluoromethyl)-3,4-dihydroisoquinolin-1-amine trifluoroacetate;
3-(3-Chlorophenyl)-3-phenyl-3,4-dihydroisoquinolin-1-amine trifluoroacetate;
3-(3′-Methoxybiphenyl-3-yl)-3-phenyl-3,4-dihydroisoquinolin-1-amine trifluoroacetate;
3-(3-Bromophenyl)-3-phenyl-3,4-dihydroisoquinolin-1-amine trifluoroacetate;
3-(3′-Methoxybiphenyl-3-yl)-3-methyl-3,4-dihydroisoquinolin-1-amine trifluoroacetate;
3-(3-Bromophenyl)-3-methyl-3,4-dihydroisoquinolin-1-amine;
3-(3-Bromophenyl)-1-(ethylthio)-3-methyl-3,4-dihydroisoquinoline;
3-Biphenyl-3-yl-3-methyl-3,4-dihydroisoquinolin-1-amine trifluoroacetate;
3-[2-(3′-Methoxybiphenyl-3-yl)ethyl]-3-methyl-3,4-dihydroisoquinolin-1-amine trifluoroacetate;
3-[2-(3-Bromophenyl)ethyl]-3-methyl-3,4-dihydroisoquinolin-1-amine trifluoroacetate;
3-[2-(3′-Methoxybiphenyl-3-yl)ethyl]-3-phenyl-3,4-dihydroisoquinolin-1-amine trifluoroacetate;
N-{[1-Amino-3-phenyl-3-(trifluoromethyl)-3,4-dihydroisoquinolin-6-yl]methyl}methanesulfonamide trifluoroacetate;
N-{[1-Amino-3-phenyl-3-(trifluoromethyl)-3,4-dihydroisoquinolin-6-yl]methyl}acetamide trifluoroacetate;
6-(Aminomethyl)-3-phenyl-3-(trifluoromethyl)-3,4-dihydroisoquinolin-1-amine bis trifluoroacetate;
3-Phenyl-6-(1H-tetrazol-5-yl)-3-(trifluoromethyl)-3,4-dihydroisoquinolin-1-amine trifluoroacetate;
1-Amino-3-phenyl-3-(trifluoromethyl)-3,4-dihydroisoquinoline-6-carboxylic acid trifluoroacetate;
1-Amino-3-phenyl-3-(trifluoromethyl)-3,4-dihydroisoquinoline-6-carbonitrile HCl salt;
1-Amino-3-(3′-methoxybiphenyl-3-yl)-3-(trifluoromethyl)-3,4-dihydroisoquinoline-6-carboxamide trifluoroacetate;
1-Amino-3-(3-bromophenyl)-3-(trifluoromethyl)-3,4-dihydroisoquinoline-6-carboxamide trifluoroacetate;
1-Amino-3-(3′-methoxybiphenyl-3-yl)-3-(trifluoromethyl)-3,4-dihydroisoquinoline-6-carboxylic acid trifluoroacetate;
1-Amino-3-(3′-methoxybiphenyl-3-yl)-3-(trifluoromethyl)-3,4-dihydroisoquinoline-6-carbonitrile trifluoroacetate;
1-Amino-3-(3-bromophenyl)-3-(trifluoromethyl)-3,4-dihydroisoquinoline-6-carbonitrile;
2-[2-(3′-methoxybiphenyl-3-yl)ethyl]-2-methyl-1,2-dihydroquinazolin-4-amine trifluoroacetate;
2-[2-(3-Bromophenyl)ethyl]-2-methyl-1,2-dihydroquinazolin-4-amine trifluoroacetate;
2-(3′-Methoxybiphenyl-3-yl)-2-methyl-1,2-dihydroquinazolin-4-amine trifluoroacetate;
2-(3-Bromophenyl)-2-methyl-1,2-dihydroquinazolin-4-amine trifluoroacetate;
4-Amino-2-[2-(3′-methoxybiphenyl-3-yl)ethyl]-2-methyl-1,2-dihydroquinazoline-7-carboxylic acid trifluoroacetate;
4-Amino-2-[2-(3-bromophenyl)ethyl]-2-methyl-1,2-dihydroquinazoline-7-carboxylic acid trifluoroacetate;
2-[2-(3′-Methoxybiphenyl-3-yl)ethyl]-1,2-dimethyl-1,2-dihydroquinazolin-4-amine trifluoroacetate,
2-[2-(3′-Methoxybiphenyl-3-yl)ethyl]-2-methyl-2H-1,3-benzoxazin-4-amine trifluoroacetate;
2-[2-(3-Bromophenyl)ethyl]-2-methyl-2H-1,3-benzoxazin-4-amine trifluoroacetate;
2-(3′-Methoxybiphenyl-3-yl)-2-methyl-2H-1,3-benzoxazin-4-amine trifluoroacetate;
2-(3-Bromophenyl)-2-methyl-2H-1,3-benzoxazin-4-amine;
2-(3-Bromophenyl)-N-methoxy-2-methyl-2H-1,3-benzoxazin-4-amine trifluoroacetate;
2-(3-Bromophenyl)-4-chloro-2-methyl-2H-1,3-benzoxazine;
2-(3-Bromophenyl)-2-methyl-2,3-dihydro-4H-1,3-benzoxazin-4-one;
3-(3′-Methoxybiphenyl-3-yl)-1′H-spiro[cyclohex-2-ene-1,2′-quinazolin]-4′-amine trifluoroacetate;
3-(3′-Methoxybiphenyl-3-yl)-1′H-spiro[cyclohexane-1,2′-quinazolin]-4′-amine trifluoroacetate;
3-Methyl-5-(trimethylsilyl)thiophene-2-carbonitrile;
5-(3-Bromophenyl)-2-(trimethylsilyl)-4,5-dihydrothieno[2,3-c]pyridin-7-amine;
5-(3-Bromophenyl)-4,5-dihydrothieno[2,3-c]pyridin-7-amine trifluoroacetate;
5-(3′-Methoxybiphenyl-3-yl)-4,5-dihydrothieno[2,3-c]pyridin-7-amine trifluoroacetate;
5-Phenyl-5-(trifluoromethyl)-2-(trimethylsilyl)-4,5-dihydrothieno[2,3-c]pyridin-7-amrine trifluoroacetate;
5-Phenyl-5-(trifluoromethyl)-4,5-dihydrothieno[2,3-c]pyridin-7-amine;
5-(3-Bromophenyl)-5-(trifluoromethyl)-2-(trimethylsilyl)-4,5-dihydrothieno[2,3-c]pyridin-7-amine trifluoroacetate;
5-(3-Bromophenyl)-5-(trifluoromethyl)-4,5-dihydrothieno[2,3-c]pyridin-7-amine trifluoroacetate;
5-(3′-Methoxybiphenyl-3-yl)-5-(trifluoromethyl)-4,5-dihydrothieno[2,3-c]pyridin-7-amine trifluoroacetate;
   or any subgroup thereof.

Compounds of the present invention also include pharmaceutically acceptable salts, alternative salts, tautomers and in vivo-hydrolysable precursors of the compounds of any of the formulas described herein. Compounds of the invention further include hydrates and solvates.

Compounds of the invention can be used as medicaments. In some embodiments, the present invention provides compounds of any of the formulas described herein, or pharmaceutically acceptable salts, tautomers or in vivo-hydrolysable precursors thereof, for use as medicaments. In some embodiments, the present invention provides compounds described herein for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides compounds of any of the formulas described herein, or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such as Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of any of the formulas described herein, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of any of the formulas described herein or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of any of the formulas described herein or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of any of the formulas described herein or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention, and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodojn) and Olanzapine/Fluoxetine (marketed as Symbyax).

In some embodiments, the mammal or human being treated with a compound of the present invention, has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipent.

When used for pharmaceutical compositions, medicaments, manufacture of a medicament, inhibiting activity of BACE, or treating or preventing Aβ-related pathologies, compounds of the present invention include the compounds of any of the formulas described herein and pharmaceutically acceptable salts, tautomers and in vivo-hydrolysable precursors thereof. Compounds of the present invention further include hydrates and solvates.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₁₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃alkyl”, whether a terminal substituent or an alkylene (or alkylenyl) group linking two substituents, is understood to specifically include both branched and straight-chain methyl, ethyl, and propyl.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms. Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 3 to 12 carbons atoms.

As used herein, “halo” or “halogen” refers to fluoro, chloro, bromo, and iodo.

“Counterion” is used to represent a small, negatively or positively charged species such as chloride (Cl⁻), bromide (Br⁻), hydroxide (OH⁻), acetate (CH₃COO⁻), sulfate (SO₄²⁻), tosylate (CH₃-phenyl-SO₃⁻), benezensulfonate (phenyl-SO₃⁻), sodium ion (Na⁺), potassium (K⁺), ammonium (NH₄⁺), and the like.

As used herein, the term “heterocyclyl” or “heterocyclic” or “heterocycle” refers to a ring-containing monovalent and divalent structures having one or more heteroatoms, independently selected from N, O and S, as part of the ring structure and comprising from 3 to 20 atoms in the rings, more preferably 3- to 7-membered rings. The number of ring-forming atoms in heterocyclyl are given in ranges herein. For example, C₅₋₁₀ heterocyclyl refers to a ring structure comprising from 5 to 10 ring-forming atoms wherein at least one of the ring-forming atoms is N, O or S. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1, 5,2-dithiazinyl, 2H-pyrrolyl, 3H-indolyl, 4-piperidonyl, 4aH-carbazole, 4H-quinolizinyl, 6H-1, 2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, xanthenyl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the term “carbonyl” is art recognized and includes such moieties as can be represented by the general formula:

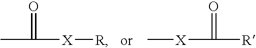

wherein X is a bond or represents an oxygen or sulfur, and R represents a hydrogen, an alkyl, an alkenyl, —(CH₂)ₘ—R″ or a pharmaceutically acceptable salt, R′ represents a hydrogen, an alkyl, an alkenyl or —(CH₂)ₘ—R″, where m is an integer less than or equal to ten, and R″ is alkyl, cycloalkyl, alkenyl, aryl, or heteroaryl. Where X is an oxygen and R and R′ is not hydrogen, the formula represents an “ester”. Where X is an oxygen, and R is as defined above, the moiety is referred to herein as a carboxyl group, and particularly when R′ is a hydrogen, the formula represents a “carboxylic acid.” Where X is oxygen, and R′ is a hydrogen, the formula represents a “formate.” In general, where the oxygen atom of the above formula is replaced by sulfur, the formula represents a “thiolcarbonyl” group. Where X is a sulfur and R and R′ is not hydrogen, the formula represents a “thiolester.” Where X is sulfur and R is hydrogen, the formula represents a “thiolcarboxylic acid.” Where X is sulfur and R′ is hydrogen, the formula represents a “thiolformate.” On the other hand, where X is a bond, and R is not a hydrogen, the above formula represents a “ketone” group. Where X is a bond, and R is hydrogen, the above formula is represents an “aldehyde” group.

As used herein, the term “sulfonyl” refers to a moiety that can be represented by the general formula:

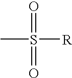

wherein R is represented by but not limited to hydrogen, alkyl, cycloalkyl, alkenyl, aryl, heteroaryl, aralkyl, or heteroaralkyl.

As used herein, some substituents are described in a combination of two or more groups. For example, the expression of “C(═O)C₃₋₉cycloalkylR^{d}” is meant to refer to a structure:

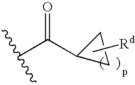

wherein p is 1, 2, 3, 4, 5, 6 or 7 (i.e., C₃₋₉cycloalkyl); the C₃₋₉cycloalkyl is substituted by R^{d}; and the point of attachment of the “C(═O)C₃₋₉cycloalkylR^{d}” is through the carbon atom of the carbonyl group, which is on the left of the expression.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

As used herein, “in vivo hydrolysable precursors” means an in vivo hydroysable (or cleavable) ester of a compound of any of the formulas described herein that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The antidementia treatment defined herein may be applied as a sole therapy or may involve, in addition to the compound of the invention, conventional chemotherapy.

Such conjoint treatment may be achieved by way of the simultaneous, sequential or separate dosing of the individual components of the treatment. Such combination products employ the compounds of this invention.

Cognitive enhancing agents memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa)

Atypical antipsychotic agents includes, but not limited to, olanzapine (marketed as Zyprexa), aripiprazole (marketed as Abilify), risperidone (marketed as Risperdal), quetiapine (marketed as Seroquel), clozapine (marketed as Clozaril), ziprasidone (marketed as Geodon) and olanzapine/fluoxetine (marketed as Symbyax).

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of any of the formulas described herein or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris(2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group which may be anionic (e.g., COOH may be COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, N⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (4CPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group C(═O)OR,

wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, C(═O)OCH₃, C(═O)OCH₂CH₃, C(═O)OC(CH₃)₃, and —C(═O)OPh. Examples of acyloxy (reverse ester) groups are represented by OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, OC(═O)CH₃ (acetoxy), OC(═O)CH₂CH₃, OC(═O)C(CH₃)₃, OC(═O)Ph, and OC(═O)CH₂Ph.

Derivatives which are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., Me, Et, -nPr, -iPr, -nBu, -sBu, -iBu, tBu); C₁₋₇aminoalkyl (e.g., aminoethyl; 2-(N,N-diethylamino)ethyl; 2(4morpholino)ethyl); and acyloxy-C₁₋₇alkyl (e.g., acyloxymethyl; acyloxyethyl; pivaloyloxymethyl; acetoxymethyl; lacetoxyethyl; 1-(1-methoxy-1-methyl)ethyl-carbonyloxyethyl; 1-(benzoyloxy)ethyl; isopropoxy-carbonyloxymethyl; 1isopropoxy-carbonyloxyethyl; cyclohexyl-carbonyloxymethyl; 1cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetahydropyranyloxy)carbonyloxyethyl; (4-tetrahydropyranyl)carbonyloxymethyl; and 1(4tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have a beneficial effects in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia. Hence compounds of the present invention and their salts are expected to be active against age-related diseases such as Alzheimer, as well as other Aβ related pathologies such as Down's syndrome and b-amyloid angiopathy. It is expected that the compounds of the present invention would most likely be used in combination with a broad range of cognition deficit enhancement agents but could also be used as a single agent.

Generally, the compounds of the present invention have been identified in one or both assays described below as having an IC₅₀ value of 100 micromolar or less.

### IGEN Assay

Enzyme is diluted 1:30 in 40 mM MES pH 5.0. Stock substrate is diluted to 12 μM in 40 mM MES pH 5.0. PALMEB solution is added to the substrate solution (1:100 dilution). DMSO stock solutions of compounds or DMSO alone are diluted to the desired concentration in 40 mM MES pH 5.0. The assay is done in a 96 well PCR plate from Nunc. Compound in DMSO (3 μL) is added to the plate then enzyme is added (27 μL) and pre-incubated with compound for 5 minutes. Then the reaction is started with substrate (30 μL). The final dilution of enzyme is 1:60; the final concentration of substrate is 6 μM (Km is 150 μM). After a 20 minute reaction at room temperature, the reaction is stopped by removing 10 μl of the reaction mix and diluting it 1:25 in 0.20M Tris pH 8.0. The compounds are added to the plate by hand then all the rest of the liquid handling is done on the CyBi-well instrument.

All antibodies and the streptavidin coated beads are diluted into PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/ml IGEN beads and a 1:5000 dilution of ruthinylated goat anti-rabbit (R^{u}-Gar) antibody is added. The final dilution of neoepitope antibody is 1:20,000, the final dilution of Ru-GAR is 1:10,000 and the final concentration of beads is 0.1 mg/ml. The mixture is read on the IGEN instrument with the CindyAB40 program after a 2-hour incubation at room temperature. Addition of DMSO alone is used to define the 100% activity. 20 μM control inhibitor is used to define 0% of control activity and 100 nM inhibitor defines 50% control of control activity in single-poke assays. Control inhibitor is also used in dose response assays with an IC50 of 100 nM.

### Fluorescent Assay

Enzyme is diluted 1:30 in 40 mM MES pH 5.0. Stock substrate is diluted to 30 μM in 40 mM MES pH 5.0. PALMEB solution is added to the substrate solution (1:100 dilution). Enzyme and substrate stock solutions are kept on ice until the placed in the stock plates. The Platemate-plus instrument is used to do all liquid handling. Enzyme (9 μL) is added to the plate then 1 μL of compound in DMSO is added and pre-incubated for 5 minutes. When a dose response curve is being tested for a compound, the dilutions are done in neat DMSO and the DMSO stocks are added as described above. Substrate (10 μL) is added and the reaction proceeds in the dark for 1 hour at room temperature. The assay is done in a Corning 384 well round bottom, low volume, non-binding surface (Corning #3676). The final dilution of enzyme is 1:60; the final concentration of substrate is 15 μM (Km of 25 μM). The fluorescence of the product is measured on a Victor II plate reader with an excitation wavelength of 360 nm and an emission wavelength of 485 nm using the protocol labeled Edans peptide. The DMSO control defines the 100% activity level and 0% activity is defined by using 50 μM of the control inhibitor, which completely blocks enzyme function. The control inhibitor is also used in dose response assays and has an IC₅₀ of 95 nM.

### Beta-Secretase Whole Cell Assay

### Generation of HEK-Fc33-1:

The cDNA encoding full length BACE was fused in frame with a three amino acid linker (Ala-Val-Thr) to the Fc portion of the human IgG1 starting at amino acid 104. The BACE-Fc construct was then cloned into a GFP/pGEN-IRES-neoK vector (a proprietary vector of AstraZeneca) for protein expression in mammalian cells. The expression vector was stably transfected into HEK-293 cells using a calcium phosphate method. Colonies were selected with 250 μg/mL of G-418. Limited dilution cloning was performed to generate homogeneous cell lines. Clones were characterized by levels of APP expression and Aβ secreted in the conditioned media using an ELISA assay developed in-house. Aβsecretion of BACE/Fc clone Fc33-1 was moderate.

### Cell Culture:

BE 93 cells stably expressing human BACE (HEK-Fc33) were grown at 37° C. in DMEM containing 10% heat-inhibited FBS, 0.5 mg/mL antibiotic-antimycotic solution, and 0.05 mg/mL of the selection antibiotic G-418.

### Aβ40 Release Assay:

Cells were harvested when between 80 to 90% confluent. 100 μL of cells at a cell density of 1.5 million/mL were added to a white 96-well cell culture plate with clear flat bottom (Costar 3610), or a clear, flat bottom 96-well cell culture plate (Costar 3595), containing 100 μL of inhibitor in cell culture medium with DMSO at a final concentration of 1%. After the plate was incubated at 37° C. for 24 h, 100 μL cell medium was transferred to a round bottom 96-well plate (Costar 3365) to quantify Aβ40 levels. The cell culture plates were saved for ATP assay as described in ATP assay below. To each well of the round bottom plate, 50 μL of detection solution containing 0.2 μg/mL of the RαAβ40 antibody and 0.25 μg/mL of a biotinylated 4G8 antibody (prepared in DPBS with 0.5% BSA and 0.5% Tween-20) was added and incubated at 4° C. for at least 7 h. Then a 50 μL solution (prepared in the same buffer as above) containing 0.062 μg/mL of a ruthenylated goat anti-rabbit antibody and 0.125 mg/mL of streptavidin coated Dynabeads was added per well. The plate was shaken at 22° C. on a plate shaker for 1 h, and then the plates were then measured for ECL counts in an IGEN M8 Analyzer. Aβ standard curves were obtained with 2-fold serial dilution of an Aβ stock solution of known concentration in the same cell culture medium used in cell-based assays.

### ATP Assay:

As indicated above, after transferring 100 μL medium from cell culture plates for Aβ40 detection, the plates, which still contained cells, were saved for cytotoxicity assays by using the assay kit (ViaLight™ Plus) from Cambrex BioScience that measures total cellular ATP. Briefly, to each well of the plates, 50 μL cell lysis reagent was added. The plates were incubated at room temperature for 10 min. Two min following addition of 100 μL reconstituted ViaLight™ Plus reagent for ATP measurement, the luminescence of each well was measured in an LJL plate reader or Wallac Topcount.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/ml in 20 mM Na Acetate pH 4.5, and then the solutions were centrifuged at 14K rpm to remove any particulates. Carboxyl groups on the dextran layer were activated by injecting a one to one mixture of 0.5M N-ethyl-N′ (3-dimethylaminopropyl)-carbodiimide (EDC) and 0.5M N-hydroxysuccinimide (NHS) at 5 μL/minute for 7 minutes. Then the stock solution of the control peptide was injected in channel 1 for 7 minutes at 5 μL/min., and then the remaining activated carboxyl groups were blocked by injecting IM ethanolamine for 7 minutes at 5 μL/minute.

### Assay Protocol:

The BACE Biacore assay was done by diluting BACE to 0.5 μM in Na Acetate buffer at pH 4.5 (running buffer minus DMSO). The diluted BACE was mixed with DMSO or compound diluted in DMSO at a final concentration of 5% DMSO. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The DMSO control was defined as 100% and the effect of the compound was reported as percent inhibition of the DMSO control.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-HERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound HERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound HERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of HERG, any offset was adjusted by determining the HERG tail current reversal potential in IonWorks™ HT, comparing it with that found in conventional electrophysiology (−82 mV; see FIG. 1c) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan HERG tail current amplitude >150 pA; post-scan seal resistance >60 MΩ. The degree of inhibition of the hERG current was assessed by dividing the post-scan hERG current by the respective pre-scan hERG current for each well.

Compounds of the present invention have been shown to inhibit beta secretase (including BACE) activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or deposition of Aβ peptide. Therefore, it is believed that the compounds of the present invention may be used for the treatment of Alzheimer disease and disease associated with dementia Hence, compounds of the present invention and their salts are expected to be active against age-related diseases such as Alzheimer, as well as other Aβ related pathologies such as Downs syndrome and β-amyloid angiopathy. It is expected that the compounds of the present invention would most likely be used as single agents but could also be used in combination with a broad range of cognition deficit enhancement agents.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described herein. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are not compatible with the reaction conditions, will be readily apparent to one skilled in the art and alternate methods must then be used.

The starting materials for the examples contained herein are either commercially available or are readily prepared by standard methods from known materials. For example the following reactions are illustrations but not limitations of the preparation of some of the starting materials and examples used herein.

General procedures for making the compounds of the invention is as follows:

The invention will now be illustrated by the following nonlimiting examples:
I. Temperatures are given in degrees Celsius (° C.); unless otherwise stated, operations were carried out at room or ambient temperature, that is, at a temperature in the range of 18-25° C.;
II. organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
III. chromatography means flash chromatography on silica gel; thin layer chromatography (TLC) was carried out on silica gel plates;
IV. in general, the course of reactions was followed by TLC or HPLC and reaction times are given for illustration only;
V. melting points are uncorrected and (dec) indicates decomposition;
VI. final products had satisfactory proton nuclear magnetic resonance (NMR) spectra;
VII. when given, NMR data is in the form of delta values for major diagnostic protons, given in parts per million (ppm) relative to tetramethylsilane (TMS) as an internal standard, determined at 300 MHz using deuterated chloroform (CDCl₃), dimethylsulphoxide (d₆-DMSO) or dimethylsulphoxide/TFA (d₆-DMSO/TFA) as solvent; conventional abbreviations for signal shape are used; for AB spectra the directly observed shifts are reported; coupling constants (J) are given in Hz; Ar designates an aromatic proton when such an assignment is made;
VIII. reduced pressures are given as absolute pressures in pascals (Pa); elevated pressures are given as gauge pressures in bars;
IX. non-aqueous reactions were run under a nitrogen atmosphere;
X. solvent ratios are given in volume:volume (v/v) terms; and
XI. Mass spectra (MS) were run using an automated system with atmospheric pressure chemical (APCI) or electrospry (+ES) ionization. Generally, only spectra where parent masses are observed are reported. The lowest mass major ion is reported for molecules where isotope splitting results in multiple mass spectral peaks (for example when chlorine is present).
XII. Commercial reagents were used without further purification.
XIII. 1-(3-Bromo-phenyl)-2,2,2-trifluoro-ethanone was prepared according to Kogon, et al, Leibigs Ann. Chem., 1992, 879-881 using NBS as the brominating agent. 1-Hydroxybenzotriazole ammonium salt was prepared according to Bajusz, et. al., FEBS Letters, 1977, 76(1), 91-2. 4-(3-Bromo-phenyl)-butan-2-one was prepared from 3-(3-bromo-phenyl)-propionic acid using standard Weinreb Amide chemistry, Nahm, et al, Tet. Lett., 1981, 3815-3818. 3-(3-Bromo-phenyl)-1-phenyl-propan-1-one was prepared from 3-(3-bromo-phenyl)-propionic acid using standard Weinreb Amide chemistry, Nahm, et al, Tet. Lett., 1981, 3815-3818. 4-Cyano-3-nitro-benzoic acid was prepared according to the procedure found in U.S. Pat. No. 2,195,076, with the exception of NMP was used in place of quinoline. 2-Methylamino-benzonitrile was prepared according to Sebastien, et al, Synlett, 2002, 164-166. 2-Hydroxy-benzamidine was prepared according to Lepore, et al, Tet. Lett. 2002, 8777-8779.
XIV. Mass spectra were recorded using either a Hewlett Packard 5988A or a MicroMass Quattro-1 Mass Spectrometer and are reported as m/z for the parent molecular ion with its relative intensity.
XV. Room temperature refers to 20-25° C.
XVI. LC-MS HPLC conditions: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm.
XVII. Agilent preparative reverse phase HPLC conditions: Compounds were purified using a Phenomenex Luna C18 reverse phase column (250×21 mm, 10 micron particle size). To one skilled in the art, it is appreciated that the crude samples can be dissolved in methanol, DMF, or a wide range of acetonitrile/water mixtures with and without TFA, methanol, or DMF in concentrations ranging from dilute to concentrated. All purifications were run using 220 nm wavelength for collecting fractions. Retention time (t_{R})=min. Agilent Gradient 1 (AG1): 0% acetonitrile with 0.1% TFA 3 min, ramp 0-50% acetonitrile/water with 0.1% TFA over 12 min, hold at 50% acetonitrile/water for 3 min, 50-100% acetonitrile/water with 0.1% TFA over 7 min, flow rate of 40 ml/min. Agilent Gradient 2 (AG2): 10-100% acetonitrile/water with 0.1% TFA over 20 min, flow rate of 40 mL/min. Agilent Gradient 3 (AG3): 0% acetonitrile with 0.1% TFA 3 min, ramp 0-100% acetonitrile/water with 0.1% TFA over 25 min, flow rate of 40 ml/min.
XVIII. Preparative reverse phase HPLC conditions: Gilson instrumentation (215 Injector, 333 Pumps and 155 U/V is Detector): Varian C8 reverse phase column (60 Angstrom irregular load in 8 mm particle size, 21 mm ID×25 cm). The crude compounds were solubilized in dimethyl sulfoxide: methanol (−1:1). Gradient elution performed with aqueous 0.1% trifluoroacetic acid/acetonitrile (typically 25-75% acetonitrile over 30 min., 95% acetonitrile over 7 min.) flow rate at 22 mL/min, UV collection at 254 nm. Retention time (t_{R})=mins. This method was used for Examples 88-94.
XIX. Normal phase chromatography conditions: Flash chromatography employed as a method for purification for selected intermediates. Isco CombiFlash Sq 16× instrument: pre-packaged disposable RediSep SiO₂ stationary phase columns (4, 12, 40, 120 gram sizes) with gradient elution at 5-125 mL/min of selected bi-solvent mixture, UV detection (190-760 nm range) or timed collection, 0.1 mm flow cell path length.
XX. Microwave heating instrumentation: A Personal Chemistry Smith Synthesizer unit (monomodal, 2.45 GHz, 300W max) was utilized for microwave heating of reactions.
XXI. Terms and abbrevations: Solvent mixture compositions are given as volume percentages or volume ratios. In cases where the NMR spectra are complex; only diagnostic signals are reported. atm: atmospheric pressure; Boc: t-butoxycarbonyl; Cbz: benzyloxycarbonyl; DCM: methylene chloride; DIPEA: diisopropylethylamine; DMF: N;N-dimethyl formamide; DMSO: dimethyl sulfoxide; Et₂O: diethyl ether; EtOAc: ethyl acetate; h: hour(s); IPLC: high pressure liquid chromatography; minute(s): min.; NMR: nuclear magnetic resonance; psi: pounds per square inch; TFA: trifluoroacetic acid; TITF: tetrahydrofuran; ACN: acetonitrile; NMP: 1-methylpyrrolidin-2-one; DMPU: 1,3-dimethyltetrahydropyrimidin-2(1H)-one; LDA: lithium diisopropylazanide

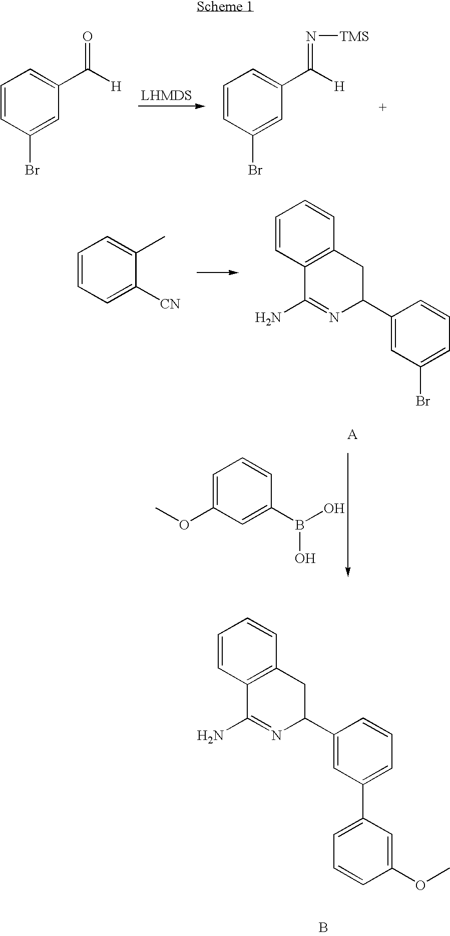

### EXAMPLE 1

### 3-(3′-Methoxybiphenyl-3-yl)-3,4-dihydroisoquinolin-1-amine trifluoroacetate (Scheme #1, B)

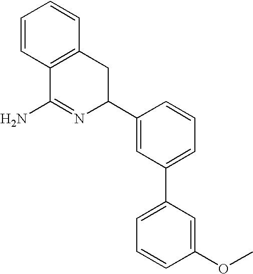

To crude 3-(3-bromophenyl)-3,4-dihydroisoquinolin-1-amine (Scheme #1, A) (100 mg, 0.332 mmol) was added cesium carbonate (325.0 mg, 0.996 mmol), 3-methoxyphenylboronic acid (53.0 mg, 0.432 mmol), dichlorobis(triphenylphosphine) palladium(II) (12.0 mg, 0.0155 mmol), and 1,2-dimethoxyethane:water:ethanol (7:3:2, 2.0 mL). The reaction was subjected to microwaves for 15 minutes at 150° C. after which the aqueous layer was removed and the organic solvents removed under reduced pressure. Acetonitrile and water were added to the brown gum, the precipitate removed, and the filtrate purified using RP-HPLC AG2 (t_{R}=9.83 min). The combined purified fractions were lyophilized to give the title compound as a TFA salt (7.9 mg, 5%). ¹H NMR (300 MHz, DMSO-d₆/TFA-d) δ 3.43-3.46 (m, 1H), 3.83-3.86 (m, 4H), 5.10 (t, J=7.1 Hz, 1H), 6.97 (dd, J=8.1, 2.0 Hz, 1H), 7.18-7.24 (m, 2H), 7.38-7.52 (m, 6H), 7.72-7.75 (m, 2H), 8.14 (d, J=7.8 Hz, 1H), m/z (APCI+) M+1 (329); t_{R}=2.18 min.

### EXAMPLE 2

### 3-(3-Bromophenyl)-3,4-dihydroisoquinolin-1-amine trifluoroacetate (Scheme #1, A)

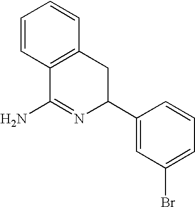

To an ice bath cooled solution of 3-bromo-benzaldehyde in THI (10 mL) was added lithium hexamethyldisilylazide 1.06M in THF (8.05 mL, 8.54 mmol) and reaction stirred cold for 2 hours. To a −78° C. cooled THF (10 mL) solution of 2-methyl-benzonitrile (1.01 mL, 8.54 mmol) and 1,3-dimethyl-tetrahydro-pyrimidin-2-one (1.55 mL, 12.80 mmol) was added 2.5M r-butyllithium in hexanes (3.41 mL, 8.54 mmol) over 5 minutes. After 20 minutes the pre-made-trimethylsilylimine was cannulated into the 2-methyl-benzonitrile anion over 10 minutes. The reaction was stirred in a −78° C. bath for 20 minutes then warmed to room temperature. After 30 minutes the reaction was quenched with 1N HCl (10 mL) and the aqueous mixture extracted three times with DCM. The organic layer was washed once with brine, dried over sodium sulfate, the solvent removed under reduced pressure, and the resulting yellow oil put under high vacuum. The bulk of the material was carried forward as is and a small portion of the crude material, 100 mg, was dissolved in acetonitrile/water and purified by RP-HPLC AG2 (t_{R}=7.8 min). The combined purified fractions were lyophilized to give the title compound as a TFA salt (25.2 mg). ¹H NMR (300 MHz, DMSO-d₆/TFA-d) δ 3.33-3.42 (m, 2H), 5.05 (t, J=7.3 Hz, 1H), 7.32-7.41 (m, 2H), 7.47 (d, J=7.5 Hz, 1H), 7.51-7.56 (m, 2H), 7.65-7.74 (m, 2H), 8.12 (d, J=7.9 Hz, 1H), m/z (APCI+) M+1 (301); t_{R}=1.89 min.

The following compounds were prepared according to scheme 1 using appropriate ketone or aldehyde starting material and boronic acid.

### EXAMPLE 3

### 3-Biphenyl-3-yl-3,4-dihydroisoquinolin-1-amine trifluoroacetate

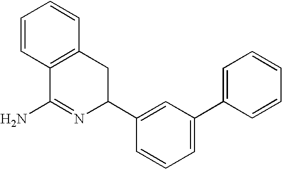

¹H NMR (300 MHz, DMSO-d₆/TFA-d) δ 3.42-3.45 (m, 2H), 5.11 (t, J=6.9 Hz, 1H), 7.37-7.42 (m, 2H), 7.46-7.57 (m, 5H), 7.65-7.74 (m, 5H), 8.14 (d, J=7.9 Hz, 1H), m/z (APCI+) M+1 (299); t_{R}=2.15 min.

### EXAMPLE 4

### 3-Phenyl-3-(trifluoromethyl)-3,4-dihydroisoquinolin-1-amine trifluoroacetate

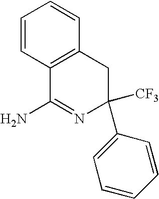

¹H NMR (300 MHz, DMSO-d₆/TFA-d) δ 3.82 (d, J=16.1 Hz, 1H), 4.15 (d, J=16.1 Hz, 1H), 7.35-7.50 (m, 4H), 7.59 (d, J=7.7 Hz, 3H), 7.70 (t, J=7.5 Hz, 1H), 8.06 (d, J=7.9 Hz, 1H), m/z (APCI+) M+1 (291); t_{R}=1.49 min.

### EXAMPLE 5

### 3-(3′-Methoxybiphenyl-3-yl)-3-(trifluoromethyl)-3,4-dihydroisoquinolin-1-amine trifluoroacetate

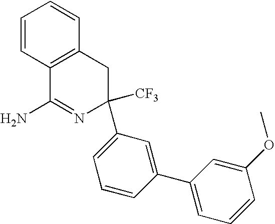

¹H NMR (300 MHz, DMSO-d₆/TFA-d) [symbol] 3.84-3.89 (m, 4H), 4.32 (d, J=16.2 Hz, 1H), 6.99 (dd, J=8.0, 2.1 Hz, 1H), 7.14-7.20 (m, 2H), 7.37-7.75 (m, 7H), 7.85 (s, 1H), 8.09 (d, J=7.9 Hz, 1H), m/z (APCI+) M+1 (397); t_{R}=2.18 min

### EXAMPLE 6

### 3-(3-Bromophenyl)-3-(trifluoromethyl)-3,4-dihydroisoquinolin-1-amine trifluoroacetate

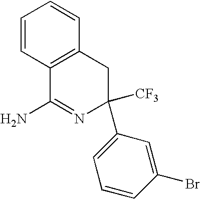

¹H NMR (300 MHz, DMSO-d₆/TFA-d) δ3.82 (d, J=16.2 Hz, 1H), 4.21 (d, J=16.2 Hz, 1H), 7.39 (t, J=8.0 Hz, 1H); 7.49 (t, J=7.5 Hz, 1H), 7.61 (d, J=7.3 Hz, 3H), 7.73 (t, J=8.1 Hz, 1H), 7.83 (s, 1H), 8.08 (d, J=7.8 Hz, 1H), m/z (APCI+) M+1 (369); t_{R}=1.90 min.

### EXAMPLE 7

### 3-(3-Chlorophenyl)-3-phenyl-3,4-dihydroisoquinolin-1-amine trifluoroacetate

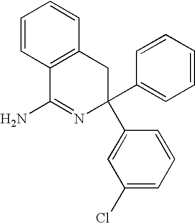

¹H NMR (300 MHz, DMSO-d₆/TFA-d) [symbol] 4.01 (s, 2H), 7.28-7.47 (m, 10H), 7.53 (d, J=7.4 Hz, 1H), 7.67 (t, J=7.5 Hz, 1H), 8.03 (d, J=7.9 Hz, 1H), m/z (APCI+) M+1 (333); t_{R}=2.03 min.

### EXAMPLE 8

### 3-(3′-Methoxybiphenyl-3-yl)-3-phenyl-3,4-dihydroisoquinolin-1-amine trifluoroacetate

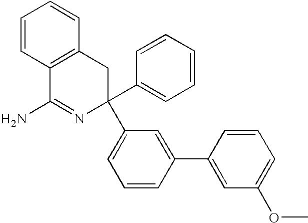

¹H NMR (300 MHz, DMSO-d₆/TFA-d) [symbol] 3.82 (s, 3H), 4.03 (d, J=16.3 Hz, 1H), 4.15 (d, J=16.2 Hz, 1H), 6.96 (dd, J=8.1, 2.0 Hz, 1H), 7.12-7.19 (m, 2H), 7.28-7.48 (m, 9H), 7.56-7.70 (m, 4H), 8.04 (d, J=7.9 Hz, 1H), m/z (APCI+) M+1 (405); t_{R}=2.39 min.

### EXAMPLE 9

### 3-(3-Bromophenyl)-3-phenyl-3,4-dihydroisoquinolin-1-amine trifluoroacetate

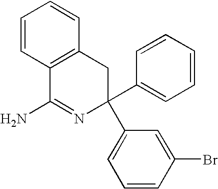

¹H NMR (300 MHz, DMSO-d₆/TFA-d) [symbol] 4.01 (s, 2H), 7.30-7.47 (m, 8H), 7.51-7.55 (m, 2H), 7.60 (s, 1H), 7.68 (t, J=7.4 Hz, 1H), 8.03 (d, J=7.8 Hz, 1H), m/z (APCI+) M+1 (377); t_{R}=2.15 min.

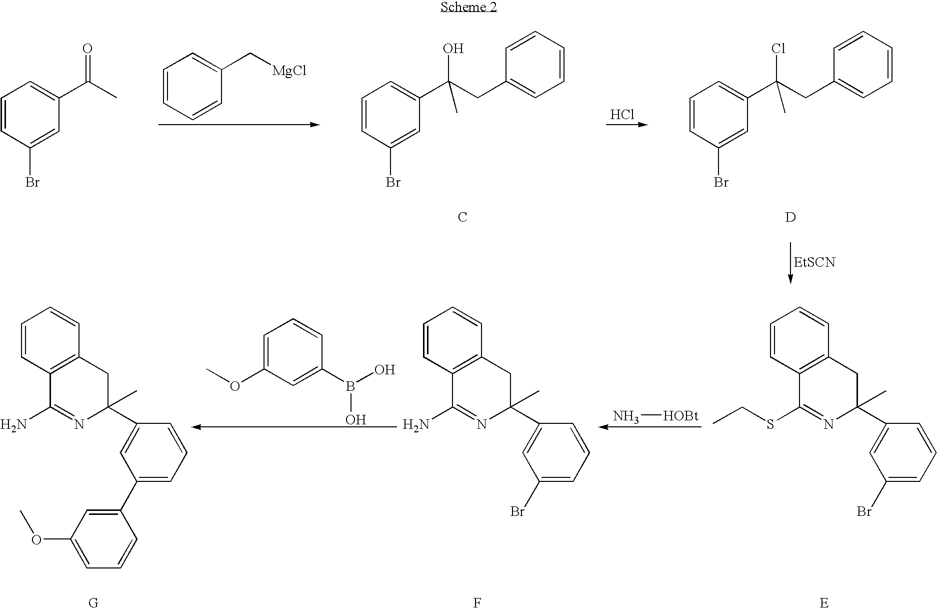

### EXAMPLE 10

### 3-(3′-Methoxybiphenyl-3-yl)-3-methyl-3,4-dihydroisoquinolin-1-amine trifluoroacetate (Scheme #2, G)

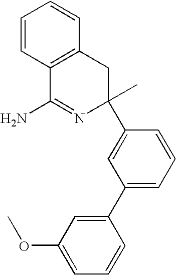

To 3-(3-bromophenyl)-3-methyl-3,4-dihydroisoquinolin-1-amine (Scheme #2, F) (50.0 mg, 0.159 mmol) was added cesium carbonate (155.0 mg, 0.476 mmol), 3-methoxyphenylboronic acid (31.0 mg, 0.206 mmol), dichlorobis(triphenylphosphine) palladium (II) (6.0 mg, 0.008 mmol), and 1,2-dimethoxyethane:water:ethanol (7:3:2, 2.0 mL). The reaction was subjected to microwaves for 15 minutes at 100° C. after which the aqueous layer was removed and the organic solvents removed under reduced pressure. Acetonitrile: water:TFA (75:25:0.1), was added to the brown gum, the precipitate removed, and the filtrate purified using RP-HPLC AG3 (t_{R}=13.6 min). The combined purified fractions were lyophilized to give the title compound as a TFA salt (40.3 mg, 56%). ¹H NMR (300 MHz, DMSO-d₆/TFA-d) δ 1.78 (s, 3H), 3.44 (d, J=16.1 Hz, 1H), 3.80-3.85 (m, 4H), 6.96 (dd, J=8.1, 2.3 Hz, 1H), 7.12-7.18 (m, 2H), 7.35-7.53 (m, 6H), 7.61-7.67 (m, 2H), 8.04 (d, J=7.8 Hz, 1H), m/z (ES+) M+1 (343); t_{R}=1.84 min.

### EXAMPLE 11

### 3-(3-Bromophenyl)-3-methyl-3,4-dihydroisoquinolin-1-amine (Scheme #2, F)

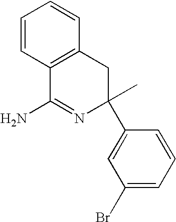

To 3-(3-bromophenyl)-1-(ethylthio)-3-methyl-3,4-dihydroisoquinoline (Scheme #2, E) (605 mg, 1.68 mmol) was added 1-hydroxybenzotriazole ammonium salt (766 mg, 5.04 mmol), and DMF (5 mL). The reaction was placed in a 100° C. bath for 5 hours. The solvent was removed under reduced pressure and the residues taken up in ethyl acetate. The organic layer was washed four times with saturated sodium bicarbonate. A white precipitate formed in the organic layer and was filtered off. The filter cake was washed with water and placed under high vacuum at 50° C. yielding the product as a white solid (145 mg, 27%). ¹H NMR (300 MHz, DMSO-d₆/TFA-d) δ 1.71 (s, 3H), 3.41 (d, J=16.2 Hz, 1H), 3.72 (d, J=16.4 Hz, 1H), 7.26 (t, J=7.9 Hz, 1H), 7.37-7.47 (m, 4H), 7.60-7.67 (m, 2H), 8.03 (d, J=7.6 Hz, 1H), m/z (APCI+) M+1 (315); t_{R}=1.87 min.

### EXAMPLE 12

### 3-(3-bromophenyl)-1-(ethylthio)-3-methyl-3,4-dihydroisoquinoline (Scheme #2, E)

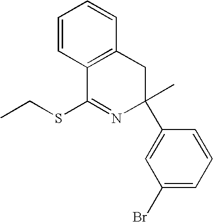

To 1-bromo-3-(1-chloro-1-methyl-2-phenylethyl)benzene (Scheme #2, D) (775 mg, 2.50 mmol) was added tin (IV) chloride (0.342 mL, 2.92 mmol) and ethyl thiocyanate (0.252-mL, 2.92 mmol). The neat reaction was placed in a 110° C. bath for 5 minutes and was quenched by adding DCM (20 mL) followed by sodium hydroxide, 1N, until the aqueous layer remained basic. The aqueous layer was removed and the organic layer dried over sodium sulfate, the solvent was removed under reduced pressure, and the orange oil put under high vacuum over night. The crude material was chromatographed on-20 g silica gel eluting with 30% DCM/hexanes. The solvent was removed from the combined fractions under reduced pressure to give the title compound as a semi-purified oil (794 mg). ¹H NMR (300 MHz, DMSO-d₆/TFA-d) δ 1.43 (t, J=7.3 Hz, 3H), 1.63 (s, 3H), 3.31-3.50 (m, 4H), 7.27-7.34 (m, 2H), 7.39-7.47 (m, 3H), 7.52-7.64 (m, 2H), 7.74-7.78 (m, 1H), m/z (ES+) M+1 (360); t_{R}=2.93 min.

### 1-Bromo-3-(1-chloro-1-methyl-2-phenylethyl)benzene (Scheme #2, D)

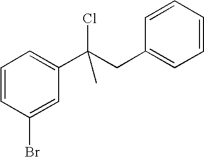

To an ice bath cooled solution 2-(3-bromophenyl)-1-phenylpropan-2-ol (Scheme #2, C) (3.70 g, 12.71 mmol) in DCM (50 mL) was inserted a Teflon tube below the surface of the solvent and anhydrous hydrogen chloride gas bubbled into the solution. After 1 hour the addition was stopped and anhydrous sodium sulfate added and filtered after 5 minutes. The solvent was removed from the filtrate under reduced pressure using a room temperature bath and the resulting oil put under high vacuum. The material was chromatographed on 75 g silica gel eluting with 30% DCM/hexanes. The solvent was removed from the combined fractions under reduced pressure without heating to give the title compound as an oil (1.12 g, 28%). ¹H NMR (300 MHz, DMSO-d₆) δ 1.90 (s, 3H), 3.43 (s, 2H), 7.00-7.03 (m, 2H), 7.19-7.23 (m, 3H), 7.33 (t, J=7.9 Hz, 1H), 7.50-7.58 (m, 2H), 7.69 (t, J=1.9 Hz, 1H)

### 2-(3-Bromophenyl)-1-phenylpropan-2-ol (Scheme #2, C)

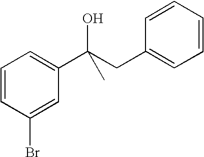

To a room temperature solution of 3-bromobenzophenone (3.32 mL, 25.12 mmol) in THF (50 mL) was added benzylmagnesium chloride 2.0M in TEF (12.60 mL, 25.20 mmol) over 5 minutes. After 2 hours the reaction was quenched with saturated ammonium chloride. Ethyl acetate was added and the aqueous layer was removed. The organic layer was washed once with saturated ammonium chloride, once with brine, dried over sodium sulfate, and the solvent removed under reduced pressure. The oil was chromatographed on 75 g silica gel eluting first with a 0-15% step gradient of DCM in hexanes (5% steps) then 100% DCM. The solvent was removed from the combined purified fractions under reduced pressure to give the title compound as an oil (3.05 g, 42%). ¹H NMR (300 MHz, 300 M , DMSO-d₆) δ 1.39 (s, 3H), 2.93 (s, 2H), 7.00-7.05 (m, 2H), 7.12-7.16 (m, 3H), 7.23 (t, J=7.9 Hz, 1H), 7.34-7.40 (m, 2H), 7.55 (t, J=−1.8 Hz, 1H).

The following compounds were prepared according to scheme #2 using the appropriate ketone starting material.

### EXAMPLE 13

### 3-Biphenyl-3-yl-3-methyl-3,4-dihydroisoquinolin-1-amine trifluoroacetate

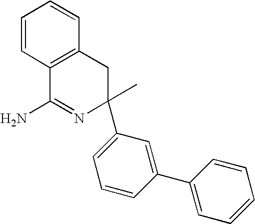

¹H NMR (300 MHz, DMSO-d₆/TFA-d) [symbol] 1.78 (s, 3H), 3.44 (d, J=16.2 Hz, 1H), 3.83 (d, J=16.3 Hz, 1H), 7.35-7.52 (m, 8H), 7.60-7.68 (m, 4H), 8.04 (d, J=7.8 Hz, 1H), m/z (ES+) M+1 (313); t_{R}=1.85 min.

### EXAMPLE 14

### 3-[2-(3′-Methoxybiphenyl-3-yl)ethyl]-3-methyl-3,4-dihydroisoquinolin-1-amine trifluoroacetate

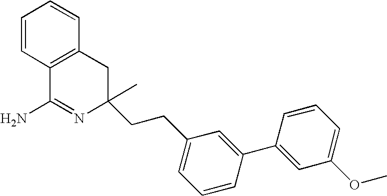

¹H NMR (300 MHz, DMSO-d₆/TFA-d) [symbol] 1.37 (s, 3H), 1.91 (t, J=7.8 Hz, 2H), 2.73 (t, J=8.3 Hz, 2H), 3.07 (d, J=16.1 Hz, 1H), 3.22 (d, J=16.2 Hz, 1H), 3.83 (s, 3H), 6.94 (dd, J=8.0, 2.1 Hz, 1H), 7.14-7.20 (m, 3H), 7.33-7.40 (m, 2H), 7.45-7.74 (m, 6H), 8.08 (d, J=7.8 Hz, 1H), m/z (APCI+) M+1 (371); t_{R}=2.27 min.

### EXAMPLE 15

### 3-[2-(3-Bromophenyl)ethyl]-3-methyl-3,4-dihydroisoquinolin-1-amine trifluoroacetate

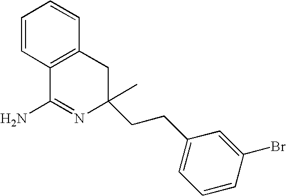

The requisite tertiary chloride intermediate to make this compound was prepared using a biphasic mixture of 1:1 saturated anhydrous zinc chloride in concentrated hydrochloric acid/chloroform. Thibblin et al, J. Am. Chem. Soc., 1977, 7926-7930. ¹H NMR (300 MHz, DMSO-d₆/TFA-d) [symbol] 1.34 (s, 3H), 1.79-1.90 (m, 2H), 2.66 (t, J=8.4 Hz, 2H), 3.04 (d, J=16.2 Hz, 1H), 3.19 (d, J=16.2 Hz, 1H), 7.17-7.26 (m, 2H), 7.34-7.55 (m, 4), 7.72 (t, J=7.5 Hz, 1H), 8.08 (d, J=7.8 Hz, 1H), m/z (APCI+) M+1 (343); t_{R}=2.12 min.

### EXAMPLE 16

### 3-[2-(3′-Methoxybiphenyl-3-yl)ethyl]-3-phenyl-3,4-dihydroisoquinolin-1-amine trifluoroacetate

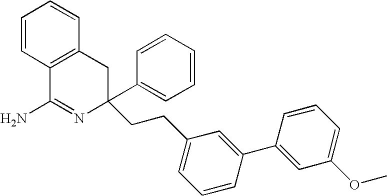

The requisite tertiary chloride intermediate to make this compound was prepared using a biphasic mixture of 1:1 saturated anhydrous zinc chloride in concentrated hydrochloric acid/chloroform. Thibblin et al, J. Am. Chem. Soc., 1977, 7926-7930. ¹H NMR (300 MHz, DMSO-d₆/TFA-d) [symbol] 2.31-2.44 (m, 2H), 2.57-2.78 (m, 2H), 3.60 (d, J=16.1 Hz, 1H), 3.77 (d, J=16.2 Hz, 1H), 3.83 (s, 3H), 6.95 (dd, J=8.2, 1.8 Hz, 1H), 7.16-7.24 (m, 4H), 7.31-7.50 (m, 10H), 7.63 (t, J=7.4 Hz, 1H), 8.02 (d, J=7.8 Hz, 1H), m/z (APCI+) M+1 (433); t_{R}=2.59 min.

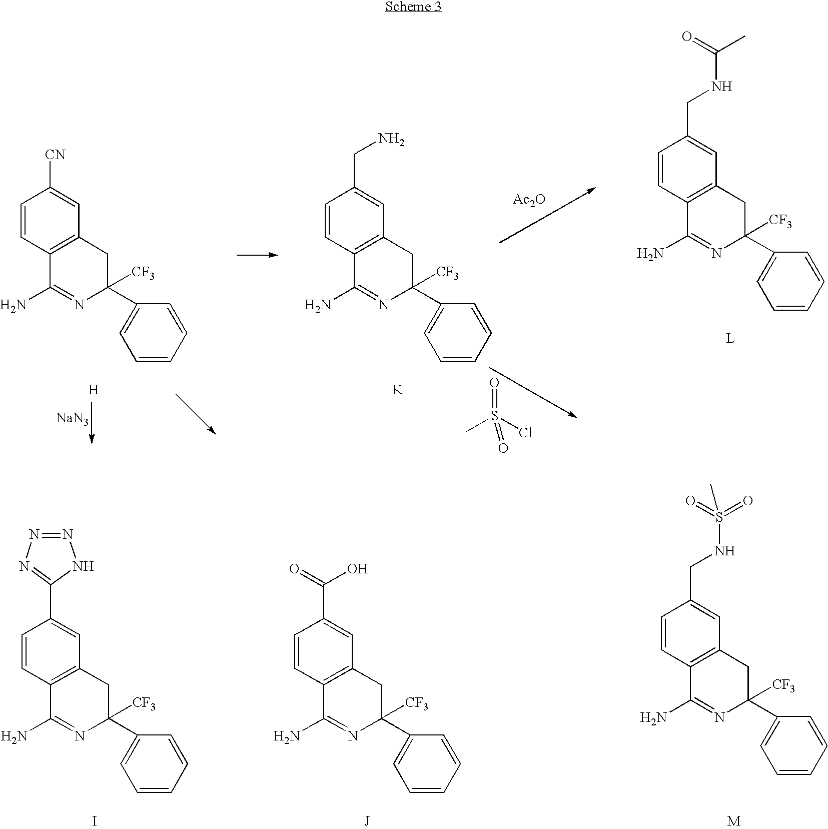

### EXAMPLE 17

### N-{[1-Amino-3-phenyl-3-(trifluoromethyl)-3,4-dihydroisoquinolin-6-yl]methyl}methanesulfonamide trifluoroacetate (Scheme #3, M)

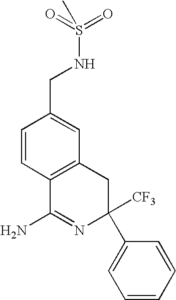

To an ice bath cooled solution of crude 6-(aminomethyl)-3-phenyl-3-(trifluoromethyl)-3,4-dihydroisoquinolin-1-amine (Scheme #3, K) (50.0 mg, 0.157 mmol) in DCM (mL) was added pyridine (15.2 uL, 0.188 mmol) and a solution of methanesulfonyl chloride (12.1 uL, 0.157 mmol) in DCM (1 mL). The reaction was warmed to room temperature and, stirred 1 hour, and the solvent removed under a stream of nitrogen. To the residue was added acetonitrile: water:TFA (75:25:0.1, 2 mL) and the mixture purified using RP-HPLC AG1 (t_{R}=12.1 min). The combined purified fractions were lyophilized to give the title compound as a TFA salt (25.2 mg, 31%). ¹H NMR (300 MHz, DMSO-d₆/TFA-d) δ 2.87 (s, 3H), 3.83 (d, J=16.2 Hz, 1H), 4.14-4.23 (m, 3H), 7.38-7.46 (m, 4H), 7.58 (t, J=7.6 Hz, 3H), 8.06 (d, J=8.2 Hz, 1H), m/z (APCI+) M+1 (398); t_{R}=1.61 min.

### EXAMPLE 18

### N-{[1-Amino-3-phenyl-3-(trifluoromethyl)-3,4-dihydroisoquinolin-6-yl]methyl}acetamide trifluoroacetate (Scheme #3, L)

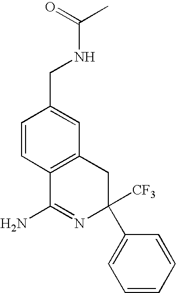

To an ice bath cooled solution of crude 6-(aminomethyl)-3-phenyl-3-(trifluoromethyl)-3,4-dihydroisoquinolin-1-amine (Scheme #3, K) (100.0 mg, 0.313 mmol) in DCM (1 mL) was added pyridine (30.3 uL, 0.376 mmol) and a solution of acetic anhydride (29.5 uL, 0.313 mmol) in DCM (1 mL). The reaction was warmed to room temperature and, stirred 20 minutes, and the solvent removed under a stream of nitrogen. To the residue was added acetonitrile:water:TFA (75:25:0.1, 2 mL) and the mixture purified using RP-HPLC AG1 (t_{R}=11.4 min). The combined purified fractions were lyophilized to give the title compound as a TFA salt (38.4 mg, 26%). ¹H NMP (300 MHz, DMSO-d₆/TFA-d) δ 1.91 (s, 3H), 3.81 (d, J=16.2 Hz, 1H), 4.13 (d, J=16.3 Hz, 1H), 4.30 (s, 2H), 7.33 (d, J=8.1 Hz, 1H), 7.38-7.47 (m, 4H), 7.59 (d, J=7.1 Hz, 2H), 8.03 (d, J=8.2 Hz, 1H), m/z (APCI+) M+1 (362); t_{R}=1.58 min.

### EXAMPLE 19

### 6-(Aminomethyl)-3-phenyl-3-(trifluoromethyl)-3,4-dihydroisoquinolin-1-amine his trifluoroacetate (Scheme #3, K)

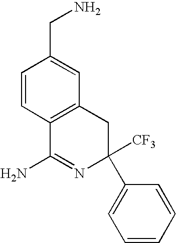

To 1-amino-3-phenyl-3-(trifluoromethyl)-3,4-dihydroisoquinoline-6-carbonitrile HCl salt (Scheme #3, H) (100.0 mg, 0.284 mmol) suspended in THF (2 mL) was added lithium aluminumhydride 1.0M in THF (1.14 mL, 1.14 mmol). After 2 hours the reaction was quenched with saturated aqueous sodium sulfate and partitioned between ethyl acetate/saturated sodium bicarbonate. The aqueous layer was removed and the organic layer dried over sodium sulfate, the solvent removed under reduced pressure, and the amber gum put under high vacuum (90 mg crude). A 40 mg portion was dissolved in acetonitrile: water:TFA (75:25:0.1, 2 mL) and purified using RP-HPLC AGI (t_{R}=9.8 min). The combined purified fractions were lyophilized to give the title compound as a bis-TFA salt (20.7 mg).

¹H NMR (300 MHz, DMSO-d₆/TFA-d) δ 3.85 (d, J=16.1 Hz, 1H), 4.08-4.13 (m, 3H), 7.39-7.47 (m, 3H), 7.54-7.59 (m, 4H), 8.13 (d, J=8.2 Hz, 1H), m/z (APCI+) M+1 (320); t_{R}=0.45 min.

### EXAMPLE 20

### 3-Phenyl-6-(1H-tetrazol-5-yl)-3-(trifluoromethyl)-3,4-dihydroisoquinolin-1-amine trifluoroacetate (Scheme #3, I)

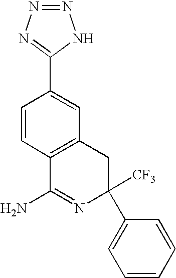

To 1-amino-3-phenyl-3-(trifluoromethyl)-3,4-dihydroisoquinoline-6-carbonitrile HCl salt (Scheme #3, H) (100.0 mg, 0.284 mmol) was added triethylamine HCl salt (117.0 mg, 0.853 mmol), sodium azide (55.0 mg, 0.853 mmol), and NMP (2.0 mL). The reaction was subjected to microwaves for 30 minutes at 150° C. The solvent was removed under reduced pressure and to the resulting gum was added acetonitrile: water and this purified using RP-HPLC AG1 (t_{R}=12.2 min). The combined purified fractions were lyophilized to give the title compound as a TFA salt (19.9 mg, 15%). ¹H NMR (300 MHz, DMSO-d₆/TFA-d) δ 3.93 (d, J=16.2 Hz, 1H), 4.33 (d, J=16.2 Hz, 1H), 7.35-7.46 (m, 3H), 7.63 (d, J=7.4 Hz, 2H), 8.11 (dd, J=8.3, 1.4 Hz, 1H), 8.28 (d, J=8.3 Hz, 1H), 8.33 (s, 1H), m/z (APCI+) M+1 (359); t_{R}=1.72 min.

### EXAMPLE 21

### 1-Amino-3-phenyl-3-(trifluoromethyl)-3,4-dihydroisoquinoline-6-carboxylic acid trifluoroacetate (Scheme #3, J)

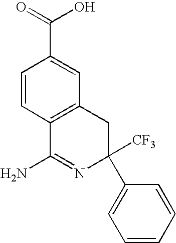

To 1-amino-3-phenyl-3-(trifluoromethyl)-3,4-dihydroisoquinoline-6-carbonitrile HCl salt (Scheme #3, H) (50.0 mg, 0.142 mmol) was added 6N HCl (2 mL) and the reaction subjected to microwaves for 15 minutes at 150° C. The solvent was removed under reduced pressure and to the resulting gum was added acetonitrile: water:TFA (75:25:0.1, 2 mL) and this purified using RP-HPLC AG1 (t_{R}=11.9 min). The combined purified fractions were lyophilized to give the title compound as a TFA salt (33.8 mg, 53%). ¹H NMR (300 MHz, DMSO-d₆/TFA-d) δ 3.87 (d, J=16.2 Hz, 1H), 4.30 (d, J=16.2 Hz, 1H), 7.35-7.46 (m, 3H), 7.60 (d, J=7.2 Hz, 2H), 7.97 (dd, J=8.2, 1.4 Hz, 1H), 8.18 (d, J=8.5 Hz, 2H), m/z (APCI+) M+1 (335); t_{R}=1.55 min.

### EXAMPLE 22

### 1-Amino-3-phenyl-3-(trifluoromethyl)-3,4-dihydroisoquinoline-6-carbonitrile HCl salt (Scheme #3, H)

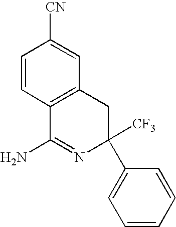

1-Amino-3-phenyl-3-(trifluoromethyl)-3,4-dihydroisoquinoline-6-carbonitrile HCl salt (Scheme #3, H) was prepared according to scheme #1 from 2,2,2-trifluoro-1-phenyl-ethanone and 2-methyl-terephthalonitrile. ¹H NMR (300 MHz, DMSO-d₆/TFA-d) δ 3.89 (d, J=16.1 Hz, 1H), 4.23 (d, J=16.3 Hz, 1H), 7.40-7.46 (m, 3H), 7.60 (d, J=7.3 Hz, 2H), 7.98 (d, J=8.2 Hz, 1H), 8.09 (s, 1H), 8.25 (d, J=8.2 Hz, 1H), m/z (ES+) M+1 (316); t_{R}=1.51 min.

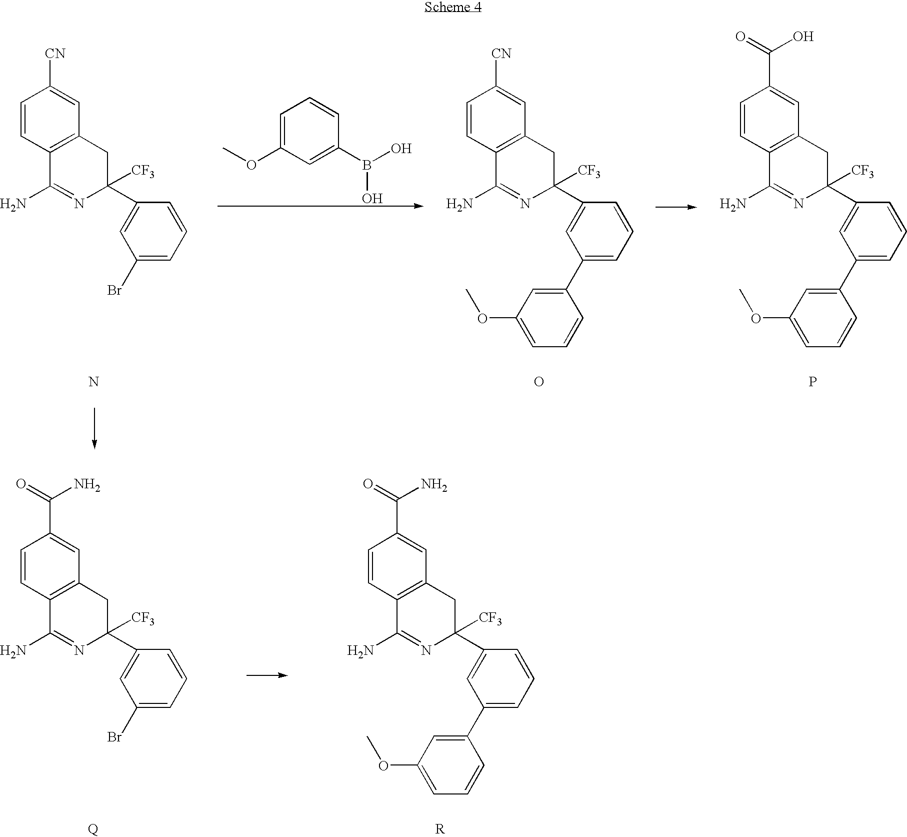

### EXAMPLE 23

### 1-Amino-3-(3′-methoxybiphenyl-3-yl)-3-(trifluoromethyl)-3,4-dihydroisoquinoline-6-carboxamide trifluoroacetate (Scheme #4, R)

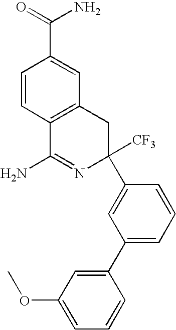

To 1-amino-3-(3-bromophenyl)-3-(trifluoromethyl)-3,4-dihydroisoquinoline-6-carboxamide TTA salt (Scheme #4, Q) (55 mg, 0.105 mmol)) was added potassium phosphate (65.0 mg, 0.307 mmol), 3-methoxyphenylboronic acid (30.0 mg, 0.200 mmol), dichlorobis(triphenylphosphine)palladium(II) (5.0 mg, 0.00667 mmol), and 1,2-dimethoxyethane:water:ethanol (7:3:2, 2.0 mL). The reaction was subjected to microwaves for 15 minutes at 100° C. after which the aqueous layer was removed and the organic solvents removed under reduced pressure. Acetonitrile and DMF were added to the brown gum, the precipitate removed, and the filtrate purified using RP-HPLC AG2 (t_{R}=8.2 min). The combined purified fractions were lyophilized to give the title compound as a TFA salt (50.7 mg, 88%). ¹H NMR (300 MHz, DMSO-d₆/TFA-d) δ 3.84 (s, 3H), 3.90 (d, J=16.1 Hz, 1H), 4.36 (d, J=16.3 Hz, 1H), 6.99 (dd, J=8.2, 2.0 Hz, 1H), 7.13-7.20 (m, 2H), 7.40 (t, J=8.0 Hz, 1H), 7.51 (t, J=7.7 Hz, 1H), 7.60 (d, J=8.0 Hz, 1H), 7.69 (d, J=7.6 Hz, 1H), 7.85-7.92 (m, 2H), 8.10-8.18 (m, 2H), m/z (APCI+) M+1 (440); t_{R}=1.91 min.

### EXAMPLE 24

### 1-Amino-3-(3-bromophenyl)-3-(trifluoromethyl)-3,4-dihydroisoquinoline-6-carboxamide trifluoroacetate (Scheme #4, Q)

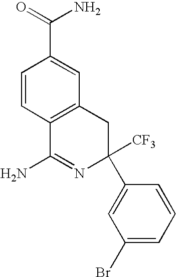

To crude 1-amino-3-(3-bromophenyl)-3-(trifluoromethyl)-3,4-dihydroisoquinoline-6-carbonitrile (Scheme #4, N) (200.0 mg, 0.507 mmol) was added toluene (2 mL) and potassium trimethylsilanolate (98.0 mg, 0.76 mmol). The reaction was subjected to microwaves for 15 minutes at 150° C. and the toluene removed under reduced pressure. Acetonitrile: water:TFA (75:25:0.1, 2 mL) was added resulting in a precipitate. To this mixture was added 2 drops TFA and the precipitate was stirred for 30 min, filtered, and put under high vacuum at 50° C. to give the product as a white TFA salt (65 mg, 24%). ¹H NMR (300 MHz, DMSO-d₆/TFA-d) δ 3.86 (d, J=16.2 Hz, 1H), 4.26 (d, J=16.5 Hz, 1H), 7.40 (t, J=8.0 Hz, 1H), 7.58-7.65 (m, 2H), 7.84 (s, 1H), 7.92 (d, J=9.4 Hz, 1H), 8.05 (s, 1H), 8.16 (d, J=8.2 Hz, 1H), m/z (APCI+) M+1 (412); t_{R}=1.62 min.

### EXAMPLE 25

### 1-Amino-3-(3′-methoxybiphenyl-3-yl)-3-(trifluoromethyl)-3,4-dihydroisoquinoline-6-carboxylic acid trifluoroacetate (Scheme #4, P)

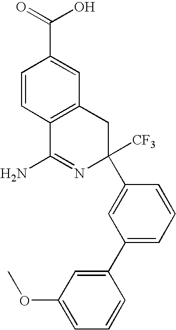

To 1-amino-3-(3′-methoxybiphenyl-3-yl)-3-(trifluoromethyl)-3,4-dihydroisoquinoline-6-carbonitrile TFA salt (Scheme #4, O) (30 mg, 0.056 mmol) was added 6N HCl (2 mL) and the reaction subjected to microwaves for 15 minutes at 150° C. The solvent was removed under reduced pressure and the resulting gum dissolved in acetonitrile/water and purified using RP-HPLC AG2 (t_{R}=8.2 min). The combined purified fractions were lyophilized to give the title compound as a TFA salt (10.5 mg, 34%). ¹H NMR (300 MHz, DMSO-d₆-/TFA-d) δ 3.84 (s, 3H), 3.91 (d, J=16.1 Hz, 1H), 4.45 (d, J=16.2 Hz, 1H), 6.99 (dd, J=8.2, 2.3 Hz, 1H), 7.13-7.21 (m, 2H), 7.40 (t, J=7.9 Hz, 1H), 7.51 (t, J=7.8 Hz, 1H), 7.60 (d, J=7.7 Hz, 1H), 7.69 (d, J=7.6 Hz, 1H), 7.98 (d, J=9.5 Hz, 1H), 8.19-8.23 (m, 2H), m/z (APCI+) M+1 (441); t_{R}=2.04 min.

### EXAMPLE 26

### 1-Amino-3-(3′-methoxybiphenyl-3-yl)-3-(trifluoromethyl)-3,4-dihydroisoquinoline-6-carbonitrile trifluoroacetate (Scheme #4, O)

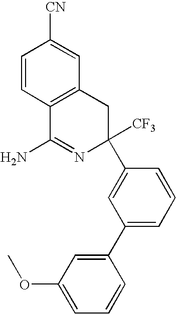

To crude 1-amino-3-(3-bromophenyl)-3-(trifluoromethyl)-3,4-dihydroisoquinoline-6-carbonitrile (Scheme #4, N) (200.0 mg, 0.507 mmol) was added cesium carbonate (496.0 mg, 1.522 mmol), 3-methoxyphenylboronic acid (93.0 mg, 0.609 mmol), dichlorobis(triphenylphosphine)palladium(II) (18.0 mg, 0.025 mmol), and 1,2-dimethoxyethane: water:ethanol (7:3:2, 2.0 mL). The reaction was subjected to microwaves for 15 minutes at 150° C. after which the aqueous layer was removed and the organic solvents removed under reduced pressure. Acetonitrile/water was added to the brown gum, the precipitate removed, and the filtrate purified using RP-HPLC AG3 (t_{R}=14.3 min). The combined purified fractions were lyophilized to give the title compound as a TFA salt (55.9 mg, 21%). ¹H NMR (300 MHz, DMSO-dr/TFA-d) δ 3.85 (s, 1H), 3.93 (d, J=16.2 Hz, 1H), 4.37 (d, J=16.3 Hz, 1H), 6.99 (dd, J=8.1, 2.2 Hz, 1H), 7.13-7.21 (m, 2H), 7.41 (t, J=8.0 Hz, 1H), 7.49-7.59 (m, 2H), 7.70 (d, J=7.4 Hz, 1H), 7.84 (s, 1H), 8.00 (d, J=8.2 Hz, 1H), 8.15 (s, 1H), 8.26 (d, J=8.2 Hz, 1H), m/z (APCI+) M+1 (422); t_{R}=2.14 min.

### EXAMPLE 27

### 1-Amino-3-(3-bromophenyl)-3-(trifluoromethyl)-3,4-dihydroisoquinoline-6-carbonitrile (Scheme #4, N)

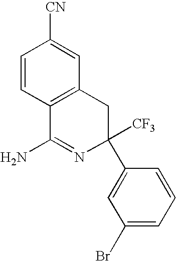

1-Amino-3-(3-bromophenyl)-3-(trifluoromethyl)-3,4-dihydroisoquinoline-6-carbonitrile (Scheme #4, N) was prepared according to scheme #1 using 2-methyl-terephthalonitrile and 1-(3-bromo-phenyl)-2,2,2-trifluoro-ethanone. ¹H NMR (300 MHz, DMSO-d₆/TFA-d) δ 3.89 (d, J=16.3 Hz, 1H), 4.26 (d, J=16.3 Hz, 1H), 7.41 (t, J=8.0 Hz, 1H), 7.62 (t, J=6.9 Hz, 2H), 7.82 (s, 1H), 8.01 (d, J=9.2 Hz, 1H), 8.09 (s, 1H), 8.25 (d, J=8.2 Hz, 1H), m/z (APCI+) M+1 (394); t_{R}=1.86 min.

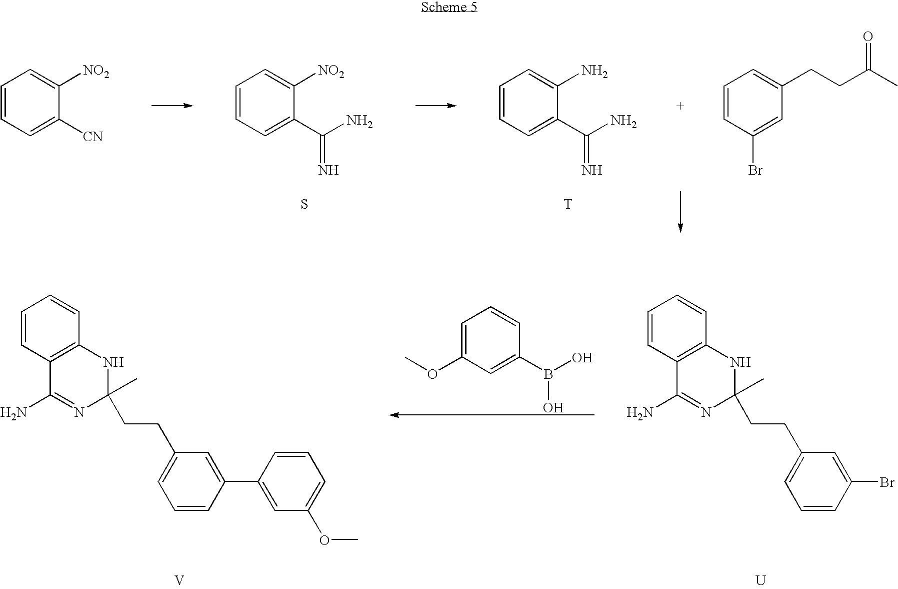

### EXAMPLE 28

### 2-[2-(3′-methoxybiphenyl-3-yl)ethyl]-2-methyl-1,2-dihydroquinazolin-4-amine trifluoroacetate (Scheme #5, V)

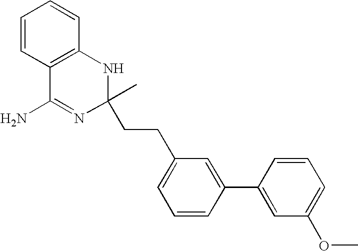

To crude 2-[2-(3-bromophenyl)ethyl]-2-methyl-1,2-dihydroquinazolin-4-amine (Scheme #5, U) (100 mg, 0.290 mmol) was added cesium carbonate (284.0 mg, 0.871 mmol), 3-methoxyphenylboronic acid (53.0 mg, 0.349 mmol), dichlorobis(triphenylphosphine) palladium(I) (10.0 mg, 0.0145 mmol), and 1,2-dimethoxyethane:water:ethanol (7:3:2, 2.0 mL). The reaction was subjected to microwaves for 15 minutes at 100° C. after which the aqueous layer was removed and the organic solvents removed under reduced pressure. Acetonitrile: water:TFA (75:25:0.1) was added to the brown gum, the precipitate removed, and the filtrate purified using RP-HPLC AG3 (t_{R}=14.3 min). The combined purified fractions were lyophilized to give the title compound as a TFA salt (42.5 mg, 30%). ¹H NMR (300 MHz, DMSO-d₆/TFA-d) δ 1.53 (s, 3H), 2.04-2.21 (m, 2H), 2.70-2.88 (m, 2H), 3.83 (s, 3H), 6.79 (t, J=8.1 Hz, 1H), 6.86 (d, J=8.1 Hz, 1H), 6.95 (dd, J=7.9, 2.2 Hz, 1H), 7.15-7.22 (m, 3H), 7.34-7.50 (m, 5H), 7.85 (d, J=8.1 Hz, 1H), m/z (APCI+) M+1 (372); t_{R}=2.22 min.

### EXAMPLE 29

### 2-[2-(3-Bromophenyl)ethyl]-2-methyl-1,2-dihydroquinazolin-4-amine trifluoroacetate (Scheme #5, U)

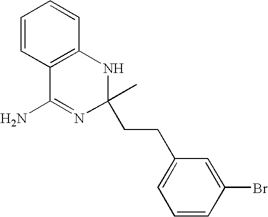

To crude 2-aminobenzenecarboximidamide HCl salt (Scheme #5, T) (1.00 g, 5.73 mmol) was added 4-(3-bromo-phenyl)-butan-2-one (0.866 g, 3.82 mmol) and ethanol (10 mL). The reaction was refluxed 18 hours and the solvent removed under reduced pressure. The bulk of the crude material was carried forward as is while a portion, 100 mg, of the crude material was dissolved in acetonitrile: water:TFA (75:25:0.1, 2 mL) and purified using RP-HPLC AG3 (t_{R}=13.1 min). The combined purified fractions were lyophilized to give the title compound as a TFA salt (57.7 mg). ¹H NMR (300 MHz, DMSO-d₆/TFA-d) δ 1.50 (s, 3H), 1.98-2.14 (m, 2H), 2.63-2.81 (m, 2H), 6.76-6.86 (m, 2H), 7.20-7.28 (m, 2H), 7.36-7.49 (m, 3H), 7.85 (d, J=8.1 Hz, 1H), m/z (APCI+) M+1 (344); t_{R}=1.98 min.

### 2-Aminobenzenecarboximidamide HCl salt (Scheme #5, T)

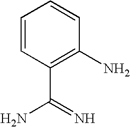

To crude 2-nitrobenzenecarboximidamide HCl salt (Scheme #5, S) (4.79 g, 23.75 mmol) was added methanol (100 mL), 10% palladium on carbon (0.5 g), and the reaction charged with hydrogen gas (50PSI). The reaction was shaken on a Parr Shaker for 20 minutes. The catalyst was filtered and the solvent removed under reduced pressure to give a tan solid which was carried forward as is (6.0 g).

### 2-Nitrobenzenecarboximidamide HCl salt (Scheme #5, S)

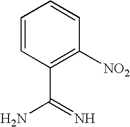

To an ice bath cooled flask containing solid 2-nitro-benzonitrile (5.00 g, 33.76 mmol) was directly added a THF solution of lithium hexamethyldisilylazide 1.0M (40.5 mL, 40.5 mmol). The reaction was stirred cold for 10 minutes then warmed to room temperature. After 1.5 hours the reaction was carefully quenched with HCl 2.0M in Et₂O (50 mL). The supernate was decanted and additional Et₂O (150 mL) was added followed by a few mL EtOAc. After triturating for 30 min the solids were filtered and partitioned between EtOAc and 1N aqueous HCl. The organic layer was washed three times with 1N HCl and the combined aqueous layers washed once with EtOAc. The aqueous solvent was removed under reduced pressure to give a brown solid which was carried forward as is. ¹H NMR (300 MHz, DMSO-d₆/TFA-d) δ 7.84 (d, J=7.4 Hz, 1H), 7.90-8.03 (m, 2H), 8.36 (d, J=8.1 Hz, 1H), m/z (ES+) M+1 (166); t_{R}=0.67 min.

The compounds below were prepared according to scheme #5 using the appropriate starting 2-nitro-benzonitrile and subsequent ketone.

### EXAMPLE 30

### 2-(3′-Methoxybiphenyl-3-yl)-2-methyl-1,2-dihydroquinazolin-4-amine trifluoroacetate

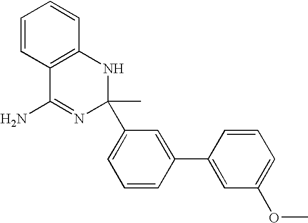

¹H NMR (300 MHz, DMSO-d₆/TFA-d) [symbol] 1.89 (s, 3H), 3.83 (s, 3H), 6.77 (t, J=8.1 Hz, 1H), 6.95-7.03 (m, 2H), 7.13-7.19 (m, 2H), 7.36-7.50 (m, 4H), 7.56-7.60 (m, 1H), 7.76-7.78 (m, 2H), m/z (APCI+) M+1 (344); t_{R}=2.04 min.

### EXAMPLE 31

### 2-(3-Bromophenyl)-2-methyl-1,2-dihydroquinazolin-4-amine trifluoroacetate

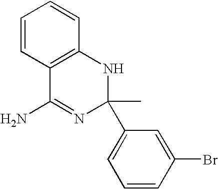

¹H NMR (300 MHz, DMSO-d₆/TFA-d) [symbol] 1.82 (s, 3H), 6.79 (t, J=7.3 Hz, 1H), 6.99 (d, J=8.1 Hz, 1H), 7.34 (t, J=7.8 Hz, 1H), 7.43-7.52 (m, 3H), 7.66 (t, J=1.7 Hz, 1H), 7.78 (d, J=8.1 Hz, 1H), m/z (APCI+) M+1 (316); t_{R}=1.72 min.

### EXAMPLE 32

### 4-Amino-2-[2-(3′-methoxybiphenyl-3-yl)ethyl]-2-methyl-1,2-dihydroquinazoline-7-carboxylic acid trifluoroacetate

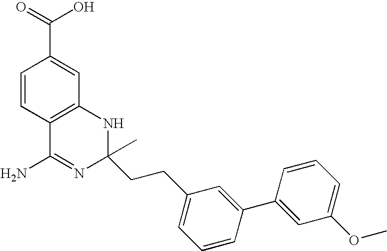

¹H NMR (300 z, DMSO-d/TFA-d) [symbol] 1.56 (s, 3H), 2.08-2.22 (m, 2H), 2.74-2.86 (m, 2H), 3.83 (s, 3H), 6.94 (d, J==8.1 Hz, 1H), 7.15-7.22 (m, 3H), 7.28 (dd, J=8.4, 1.6 Hz, 1H), 7.37 (t, J=7.8 Hz, 2H), 7.45-7.50 (m, 3H), 7.97 (d, J=8.3 Hz, 1H), m/z (APCI+) M+1 (416); t_{R}=2.13 min.

### EXAMPLE 33

### 4-Amino-2-[2-(3-bromophenyl)ethyl]-2-methyl-1,2-dihydroquinazoline-7-carboxylic acid trifluoroacetate

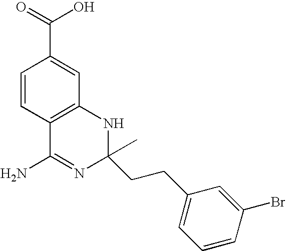

¹HNMR (300 MHz, DMSO-d₆/TFA-d) [symbol] 1.53 (s, 3H), 2.00-2.15 (m, 1H), 2.65-2.79 (m, 1H), 7.20-7.30 (m, 3H), 7.36-7.44 (m, 3H), 7.97 (d, J=8.4 Hz, 1H), m/z (APCI+) M+1 (388); t_{R}=1.87 min.

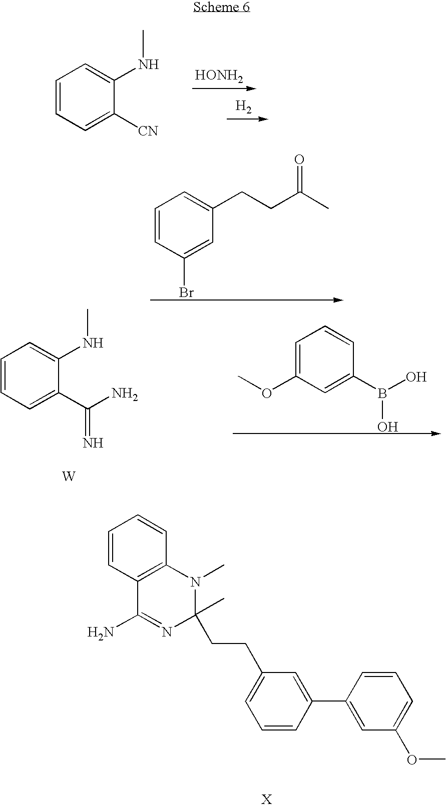

### EXAMPLE 34

### 2-[2-(3′-Methoxybiphenyl-3-yl)ethyl]-1,2-dimethyl-1,2-dihydroquinazolin-4-amine trifluoroacetate (Scheme #6, X)

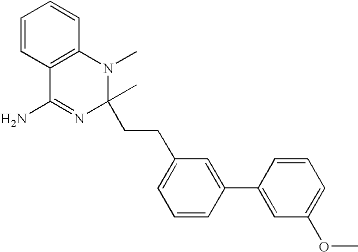

To crude 2-(methylamino)benzenecarboximidamide (113 mg, 0.757 mmol) (Scheme #6, W) was added NMP (2.0 mL) followed by 4-(3-bromo-phenyl)-butan-2-one (172 mg, 0.757 mmol) and the reaction subjected to microwaves for 30 min at 200° C. The NMP was removed under reduced pressure and to the crude mixture was added 3-methoxyphenylboronic acid (172 mg, 1.36 mmol), cesium carbonate (740 mg, 2.27 mmol), dichlorobis(triphenylphosphine) palladium (II) (27 mg, 0.0379 mmol), and 1,2-dimethoxyethane:water:ethanol (7:3:2, 2.0 mL). The reaction was subjected to microwaves for 15 minutes at 100° C. after which the aqueous layer was removed and the organic solvents removed under reduced pressure. Acetonitrile: water:TFA (75:25:0.1), was added to the brown gum, the precipitate removed, and the filtrate purified using RP-HPLC AG1 (t_{R}=17.8 min). The combined purified fractions were lyophilized to give the title compound as a TFA salt (3.6 mg, 1%). ¹H NMR (300 MHz, DMSO-d₆/TFA-d) [symbol] 1.53 (s, 3H), 2.04-2.15 (m, 1H), 2.30-2.45 (m, 1H), 2.67-2.80 (m, 2H), 2.95 (s, 3H), 3.83 (s, 3H), 6.89-7.02 (m, 3H), 7.16-7.28 (m, 3H), 7.37 (t, J=7.7 Hz, 2H), 7.47-7.53 (m, 2H), 7.61 (t, J=7.3 Hz, 1H), 7.93 (d, J=6.2 Hz, 1H), m/z (ES+) M+1 (386); t_{R}=2.14 min

### 2-(Methylamino)benzenecarboximidamide (Scheme #6, W)

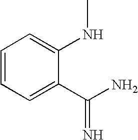

To 2-Methylamino-benzonitrile (100 mg, 0.757 mmol) was added potassium hydroxide (127 mg, 2.27 mmol), hydroxylamine hydrochloride (1.05 mg. 1.51 mmol), and methanol (2.0 mL). The reaction refluxed for 18 hours after which the solvent was removed under reduced pressure and the residues triturated with 10:1:1 EtOAc/DCM/MeOH. The salts were filtered off and the solvent removed from the filtrate under reduced pressure. To the brown solid was added EtOH (5 mL) and an unweighed amount of Raney Nickel previously washed with EtOH. The reaction was charged with hydrogen gas (50 PSI), heated to 60° C., and shaken on a Parr Shaker for 18 hours. The catalyst was removed and the solvent removed from the filtrate under reduced pressure to give a greenish gum which was used as is into the next reaction. m/z (ES+) M+1 (150); t_{R}=0.36 min

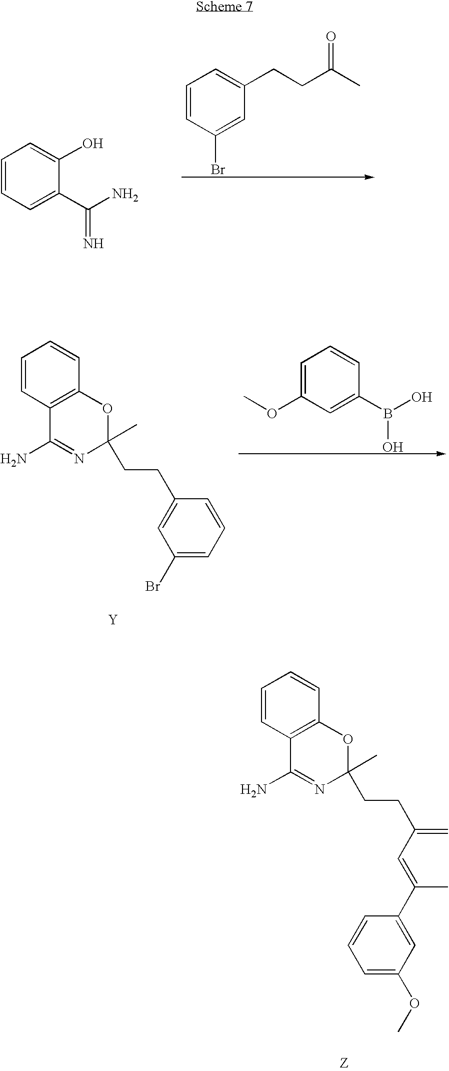

### EXAMPLE 35

### 2-[2-(3-Methoxybiphenyl-3-yl)ethyl]-2-methyl-2H-1,3-benzoxazin-4-amine trifluoroacetate (Scheme #7, Z)

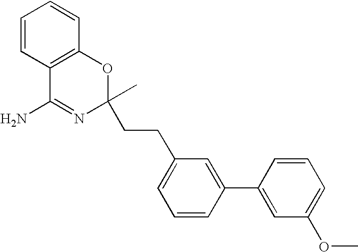

To -[2-(3-bromophenyl)ethyl]-2-methyl-2H-1,3-benzoxazin-4-amine TFA salt (45 mg, 0.098 mmol) (Scheme #7, Y) was added cesium carbonate (96 mg, 0.29 mmol), 3-methoxyphenylboronic acid (22 mg, 0.15 mmol), dichlorobis(triphenylphosphine)palladium(II) (3.4 mg, 0.0049 mmol), and 1,2-dimethoxyethane: water:ethanol (7:3:2, 2.0 mL). The reaction was subjected to microwaves for 15 minutes at 100° C. after which the aqueous layer was removed and the organic solvents removed under reduced pressure. Acetonitrile: water:TFA (75:25:0.1) (2.0 mL) was added to the brown gum, the precipitate removed, and the filtrate purified using RP-HPLC AG1 (t_{R}=16.7 min). The combined purified fractions were lyophilized give the title compound as a TFA salt (48 mg, 101%). ¹H NMR (300 MHz, DMSO-d₆-/TFA-d) 61.69 (s, 3H), 2.29 (t, J=8.3 Hz, 2H), 2.79-2.90 (m, 2H), 3.83 (s, 3H), 6.94 (dd, J=7.8, 2.2 Hz, 1H), 7.15-7.22 (m, 4H), 7.29 (t, J=8.1 Hz, 1H), 7.37 (td, J=7.9, 2.1 Hz, 2H), 7.47-7.50 (m, 2H), 7.74 (dd, J=15.7, 1.4 Hz, 1H), 8.10 (dd, J=8.0, 1.3 F 1H); m/z (APCI+) M+1 (373); t_{R}=2.38 min.

### EXAMPLE 36

### 2-[2-(3-Bromophenyl)ethyl]-2-methyl-2H-1,3-benzoxazin-4-amine trifluoroacetate (Scheme #7, Y)

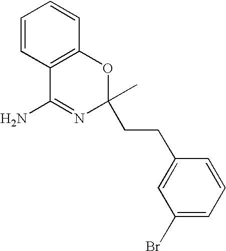

To 2-Hydroxy-benzamidine (600 mg, 4.41 mmol) was added 4-(3-bromo-phenyl)-butan-2-one (1.00 g, 4.41 mmol), p-toluenesulfonic acid monohydrate (84 mg, 0.44 mmol), and toluene (15 mL). The reaction was fitted with a prefilled Dean-Stark trap and heated to reflux. After refluxing overnight the solvent was removed under reduced pressure and the solids put under high vacuum. Et₂O was added and the solids triturated for 1 hour and removed. The filtrate was removed of solvent under reduced pressure, redissolved in ACN, and purified using RP-HPLC AG1 (t_{R}=15.5 min). The combined purified fractions were lyophilized to give the title compound as a TFA salt (45 mg, 3%). m/z (ES+) M+1 (345); t_{R}=1.88 min

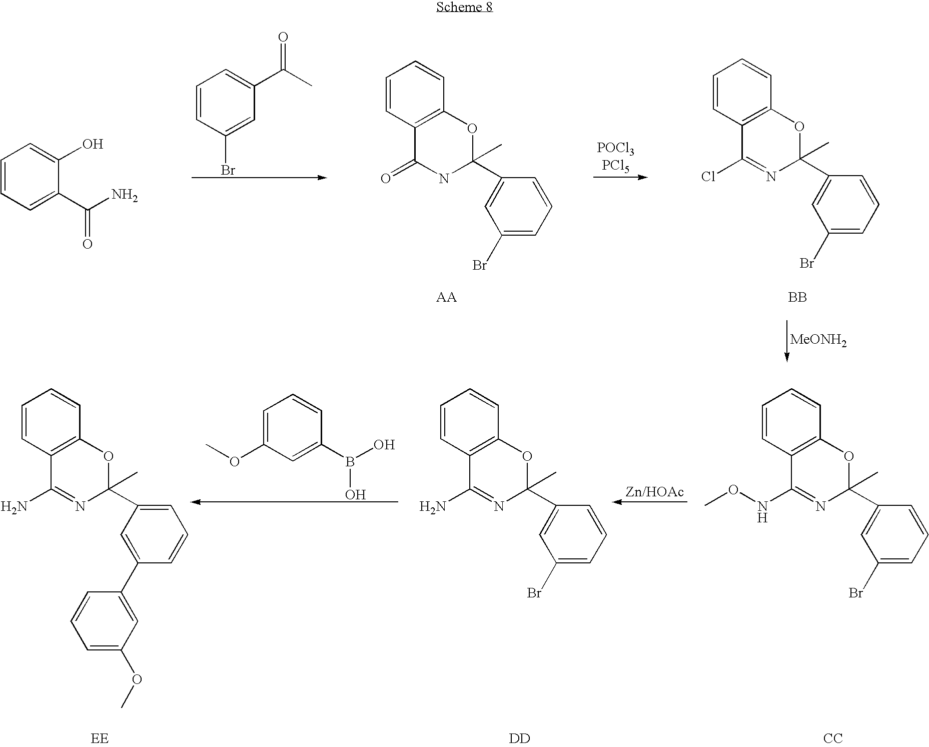

### EXAMPLE 37

### 2-(3′-Methoxybiphenyl-3-yl)-2-methyl-2H-1,3-benzoxazin-4-amine trifluoroacetate (Scheme #8, EE)

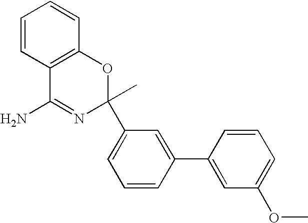

To crude 2-(3-bromophenyl)-2-methyl-2H-1,3-benzoxazin-4-amine (Scheme #8, DD) (70 mg, 0.162 mmol) was added cesium carbonate (211 mg, 0.216 mmol), 3-methoxyphenylboronic acid (49 mg, 0.32 mmol), dichlorobis(triphenylphosphine)palladium (II) (7.6 mg, 0.011 mmol), and 1,2-dimethoxyethane: water:ethanol (7:3:2, 2.0 mL). The reaction was subjected to microwaves for 15 minutes at 100° C. after which the aqueous layer was removed and the organic solvents removed under reduced pressure. Acetonitrile:water:TFA (75:25:0.1) (2.0 mL) was added to the brown gum, the precipitate removed, and the filtrate purified using RP-HPLC AG1 (t_{R}=15.6 min). The combined purified fractions were lyophilized to give the title compound as a TFA salt (20 mg, 27%). ¹H NMR (300 MHz, DMSO-d₆/TFA-d) δ2.05 (s, 3H), 3.83 (s, 3H), 6.97 (dd, J=8.1, 2.4 Hz, 1H), 7.11-7.22 (m, 3H), 7.35-7.49 (m, 4H), 7.59-7.64 (m, 1H), 7.70-7.75 (m, 2H), 7.99 (dd, J=8.0, 1.3 Hz, 1H); m/z (APCI+) M+1 (345); t_{R}=2.13 min.

### EXAMPLE 38

### 2-(3-Bromophenyl)-2-methyl-2H-1,3-benzoxazin-4-amine (Scheme #8, DD)

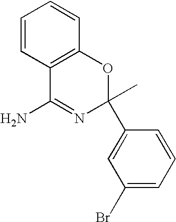

To impure 2-(3-bromophenyl)-N-methoxy-2-methyl-2H-1,3-benzoxazin-4-amine TFA salt (Scheme #8, CC) (75 mg, 0.162 mmol) was added acetic acid (1.5 mL) and powdered zinc (28 mg, 0.432 mmol). The reaction was stirred for 1 hour, the zinc filtered off and the acetic acid removed under reduced pressure. The solid was used as is in the next reaction. m/z (APCI+) M+1 (317); t_{R}=1.95 min.

### EXAMPLE 39

### 2-(3-Bromophenyl)-N-methoxy-2-methyl-2H-1,3-benzoxazin-4-amine trifluoroacetate (Scheme #8, CC)

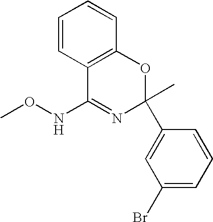

To 2-(3-bromophenyl)-4-chloro-2-methyl-2H-1,3-benzoxazine (Scheme #8, BB) (100 mg, 0.297 mmol) in DMF (1.0 mL) was added DIPEA (0.26 mL, 1.49 mmol) and methoxyamine hydrochloride (124 mg, 1.49 mmol). The reaction was placed in a 100° C. bath for 10 hours and the DMF was removed under reduced pressure. The crude mixture was dissolved in acetonitrile:water:TFA (75:25:0.1) (4.0 mL) and purified using RP-HPLC AG2 (t_{R}=16.4 and 17.9 min). Two peaks with the same molecular weight were collected, combined, and lyophilized to give the title compound as a TFA salt (78 mg, 57%). ¹H NMR (300 MHz, DMSO-d₆/TFA-d) [symbol] 1.78 (s, 3H), 3.86 (s, 2.5H), 3.93 (s, 0.5H), 6.86 (t, J=8.2 Hz, 1H), 7.01 (d, J=7.7 Hz, 1H), 7.26 (d, J=7.9 Hz, 1H), 7.37-7.42 (m, 2H), 7.52-7.54 (m, 2H), 8.03 (s, 1H); m/z (ES+) M+1 (348)

### EXAMPLE 40

### 2-(3-Bromophenyl)-4-chloro-2-methyl-2H-1,3-benzoxazine (Scheme #8, BB)

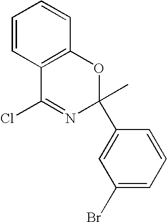

To 2-(3-bromophenyl)-2-methyl-2,3-dihydro-4H-1,3-benzoxazin-4-one (Scheme #8, AA) (5.00 g, 15.71 mmol) was added phosphorous (III) oxychloride (8.8 mL, 94.28 mmol) and phosphorous (V) chloride (0.33 g, 1.57 mmol). The reaction was placed in a 50° C. bath and stirred for 2 hours. Additional phosphorous (V) chloride (0.33 g, 1.57 mmol) was added and the reaction stirred 1 hour. Any remaining phosphorous (III) oxychloride was removed under reduced pressure and to the resulting oil was added DCM/hexanes (1:1, 25 mL). This solution was applied to 600 mL silica gel and eluted with DCM/hexanes (1:1). The combined purified fractions were removed of solvent under reduced pressure to give the title compound as a pale oil (3.37 g, 64%). ¹H NMR (300 MHz, DMSO-d₆) [symbol] 1.86 (s, 3H), 7.10-7.14 (m, 2H), 7.36 (t, J=7.9 Hz, 1H), 7.53-7.60 (m, 4H), 7.68 (t, J=1.8 Hz, 1H); m/z (ES+) M+1 (336); t_{R}=2.75 min.

### EXAMPLE 41

### 2-(3-Bromophenyl)-2-methyl-2,3-dihydro-4H-1,3-benzoxazin-4-one (Scheme #8, AA)

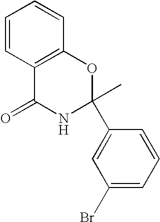

To salicylamide (10.00 g, 72.92 mmol) in toluene (50 mL) was added 3-bromoacetophenone (14.6 mL, 109.38 mmol) and p-toluenesulfonic acid monohydrate (1.39 g, 7.29 mmol). The reaction was fitted with a prefilled Dean-Stark trap and refluxed over night. The reaction was cooled to room temperature then in an ice bath for 30 minutes. The resulting precipitate was filtered, washed with toluene, and put under high vacuum at 75° C. for 4 hours to give the title compound as a white solid (18.82 g, 81%). ¹H NMR (300 MHz, DMSO-d₆/TFA-d) [symbol] 1.79 (s, 3H), 7.01 (dd, J=15.0, 0.9 Hz, 1H), 7.10 (d, J=7.8 Hz, 1H), 7.45 (d, J=8.0 Hz, 3H), 7.60-7.62 (m, 2H), 7.65 (d, J=1.7 Hz, 1H); m/z (ES+) M+1 (318); t_{R}=2.13 min.

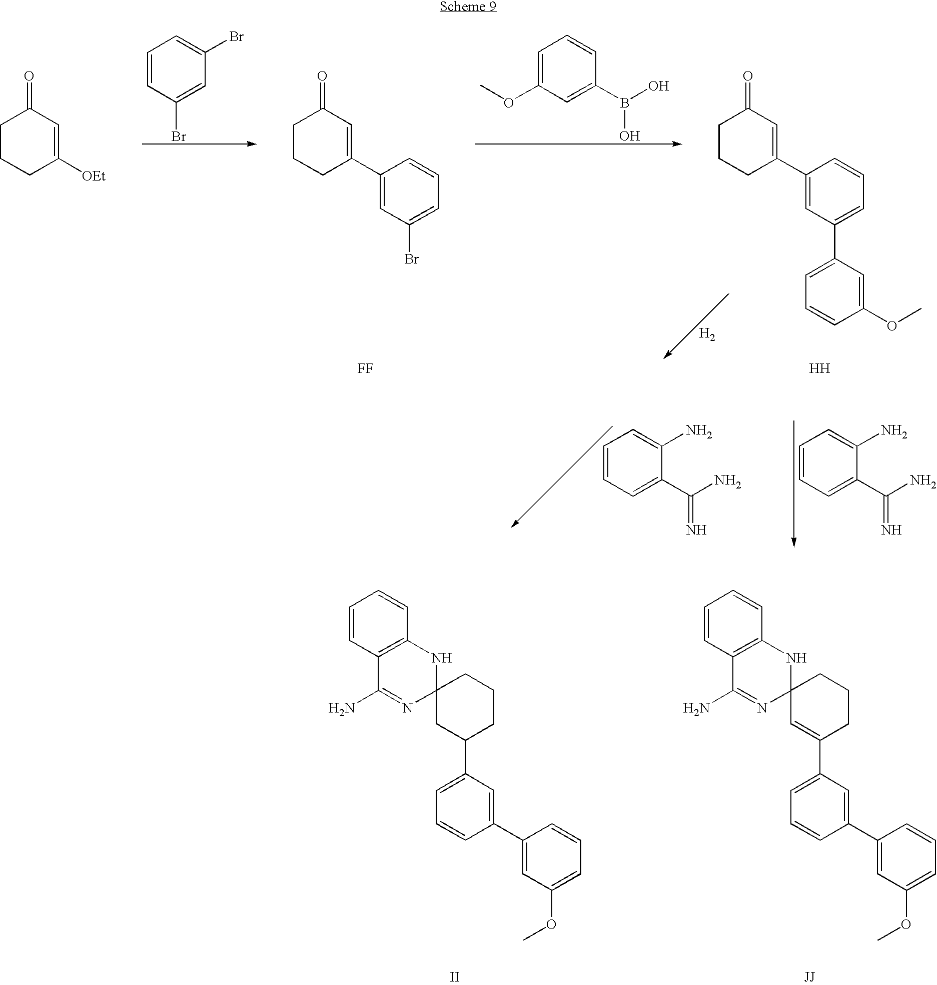

### EXAMPLE 42

### 3-(3′-Methoxybiphenyl-3-yl)-1′H-spiro[cyclohex-2-ene-1,2′-quinazolin]-4′-amine trifluoroacetate (Scheme #9, JJ)

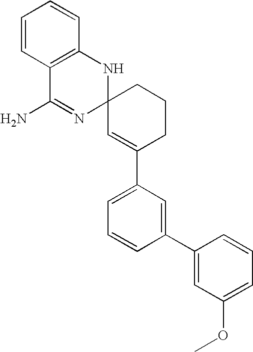

To 3-(3′-methoxybiphenyl-3-yl)cyclohex-2-en-1-one (Scheme #9, HH) (100 mg, 0.36 mmol) was added crude 2-amino-benzamidine HCl salt (94 mg, 0.54 mmol) and EtOH (2.0 mL). The reaction was subjected to microwaves for 15 minutes at 100° C. followed by 15 minutes at 150° C. The solvent was removed under reduced pressure, the residue dissolved in acetonitrile: water:TFA (75:25:0.1) (2.0 mL), and purified using RP-HPLC AG2 (t_{R}=12.2 min). The combined purified fractions were lyophilized to give the title compound as a TFA salt (51 mg, 28%). ¹H NMR (300 MHz, DMSO-d₆/TFA-d) [symbol] 1.85-1.97 (m, 1H), 2.02-2.14 (m, 1H), 2.38-2.46 (m, 1H), 2.54-2.61 (m, 2H), 2.96 (dd, J=30.4, 17.4 Hz, 1H), 3.84 (s, 3H), 6.36 (d, J=20.0 Hz, 1H), 6.79-6.97 (m, 3H), 7.21-7.27 (m, 2H), 7.37-7.74 (m, 6H), 7.87 (dd, J=7.0, 3.7 Hz, 1H); m/z (ES+) M+1 (396); t_{R}=2.11 min.

### 3-(3′-Methoxybiphenyl-3-yl)cyclohex-2-en-1-one (Scheme #9, HH)

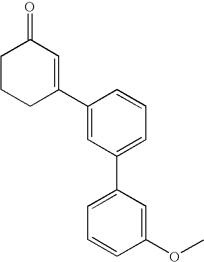

To 3-(3-bromophenyl)cyclohex-2-en-1-one (Scheme #9, FF) (3.00 g 11.95 mmol) was added potassium phosphate (5.07 g, 23.89 mmol), 3-methoxyphenylboronic acid (2.18 g, 14.34 mmol), dichlorobis(triphenylphosphine)palladium(II) (0.42 g, 0.60 mmol), and 1,2-dimethoxyethane: water:ethanol (7:3:2, 10.0 mL). The reaction was heated in a J-Kem block at 80° C. for 1 hour. The aqueous layer was removed and the organic solvent removed under reduced pressure. To the resulting brown oil was added 30% EtOAc/hexanes and the solution applied to 50 g silica gel eluting with the same solvent system. The combined purified fractions were removed of solvent under reduced pressure to give the title compound as a yellow oil (3.25 g, 98%). ¹H NMR (300 MHz, DMSO-d₆) [symbol] 2.07 (quintet, J=6.3 Hz, 2H), 2.40 (t, J=6.7 Hz, 2H), 2.85 (t, J=6.6 Hz, 2H), 3.84 (s, 3H), 6.45 (s, 1H), 6.96 (ddd, J=8.1, 2.5, 0.9 Hz, 1H), 7.25-7.30 (m, 2H), 7.39 (t, J=8.1 Hz, 1H), 7.53 (t, J=7.7 Hz, 1H), 7.63-7.67 (m, 1H), 7.72-7.75 (m, 1H), 7.86 (t, J=1.7 Hz, 1H); m/z (APCI+) M+1 (279); t_{R}=2.65 min.

### 3-(3-Bromophenyl)cyclohex-2-en-1-one (Scheme #9, FF)

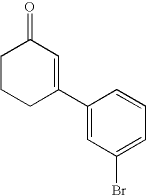

To a −78° C. cooled solution of 1,3-dibromobenzene (10.3 mL, 84.8 mmol) in THF (200 mL) was added 2.5M n-butyllithium (33.9 mL, 84.8 mmol) over 10 minutes. After stirring cold for 10 minutes, 3-ethoxy-cyclohex-2-enone (18.5 mL, 127.2 mmol) in THF (30 mL) was added dropwise over 5 minutes. After stirring cold for 30 minutes the reaction was warmed to room temperature and quenched with water (590 mL). The mixture was partitioned between Et₂O/saturated NaCl and the aqueous layer removed. The organic layer was washed three times with saturated NaCl, dried over MgSO₄, the solvent removed under reduced pressure, and the residue put under high vacuum to give the product as a yellow oil. (18.83 g, 88%). ¹H NMR (300 MHz, DMSO-d₆) [symbol] 2.04 (quintet, J=6.1 Hz, 2H), 2.38 (t, J=6.3 Hz, 2H), 2.76 (t, J=6.0 Hz, 2H), 6.36 (d, J=1.4 Hz, 1H), 7.41 (td, J=7.9, 1.2 Hz, 1H), 7.65 (td, J=7.0, 1.0 Hz, 2H), 7.82 (d, J=1.6 Hz, 1H); m/z (APCI+) M+1 (251); t_{R}=2.36 min.

### EXAMPLE 43

### 3-(3′-Methoxybiphenyl-3-yl)-1′H-spiro[cyclohexane-1,2′-quinazolin]-4′-amine trifluoroacetate (Scheme #9, II)

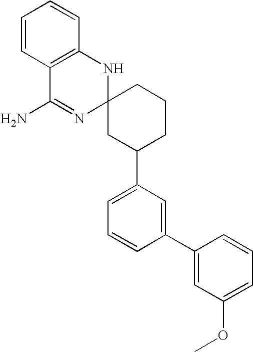

To 3-(3′-methoxybiphenyl-3-yl)cyclohex-2-en-1-one (Scheme #9, HH) (50 mg, 0.18 mmol) in MeOH (5 mL) was added 10% Pd/C (10 mg) and the reaction charged with H₂ (50 PSI). After shaking on a Parr shaker for 1.5 hours the catalyst was filtered off and the organic solvent was removed under reduced pressure. To the resulting residue was added crude 2-amino-benzamidine HCl salt (75 mg, 0.43 mmol) and EtOH (2.0 mL). The reaction was subjected to microwaves for 20 minutes at 150° C. The solvent was removed under reduced pressure and the residue dissolved in acetonitrile:water:TFA (75:25:0.1) (2.0 mL) and purified using RP-HPLC AG2 (t_{R}=13.0 min). The combined purified fractions were lyophilized to give the title compound as a TFA salt (19 mg, 21%). ¹H NMR (300 MHz, DMSO-d₆/TFA-d) [symbol] 1.51-1.96 (m, 6H), 2.14-2.37 (m, 2H), 2.93-3.15 (m, 1H), 3.83 (s, 3H), 6.77-6.87 (m, 2H), 6.95 (dd, J=8.1, 2.3 Hz, 1H), 7.11-7.25 (m, 3H), 7.35-7.52 (m, 5H), 7.84 (d, J=8.3 Hz, 1H); m/z (APCI+) M+1 (398); t_{R}=2.45 min.

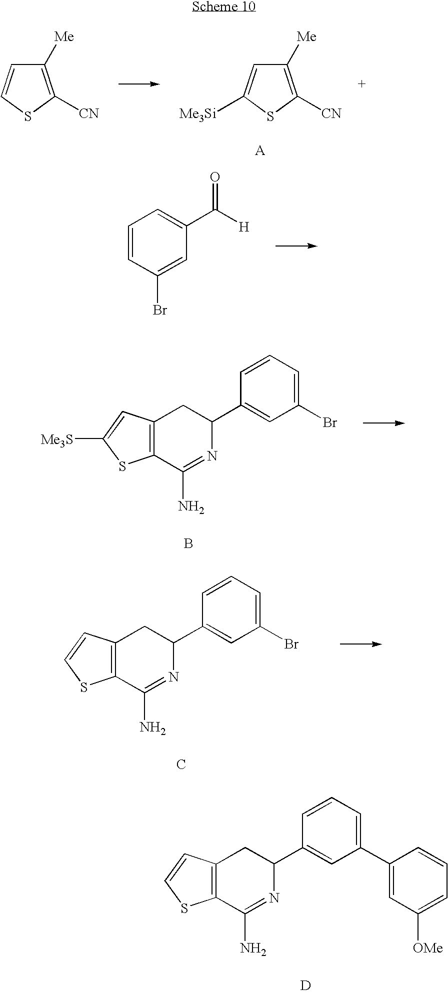

### EXAMPLE 44

### 3-Methyl-5-(trimethylsilyl)thiophene-2-carbonitrile (Scheme #10, A)

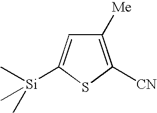

To a −78° C. stirred solution of freshly prepared LDA (2.17 g, 20.30 mmol) in THF (20 mL) was slowly added 3-methylthiophene-2-carbonitrile (2.50 g, 20.30 mmol) in THF (10 mL) and the reaction was stirred at −78° C. for 5 minutes. To this anion was slowly added trimethylsilyl chloride (2.84 mL, 22.33 mmole) and the reaction stirred at −78° C. for 30 minutes. The ice bath was removed, warmed to room temperature and stirred an additional hour. The THF was removed under reduced pressure at ambient temperature to yield a bright yellow oil. The crude compound was purified using flash chromatography (neutral activated alumina, 10:90 ether:hexanes) to give the title compound as a volatile, clear colorless oil (2.52 g, 64%). ¹H NMR (300 MHz, DMSO-d₆): δ 0.34 (s, 9H); 2.43 (s, 3H); 6.95 (s, 1H). HPLC (Platform 3): 2.93 minutes. m/z (APCI) 237 M+41.

### EXAMPLE 45

### 5-(3-Bromophenyl)-2-(trimethylsilyl)-4,5-dihydrothieno[2,3-c]pyridin-7-amine (Scheme #10, B)

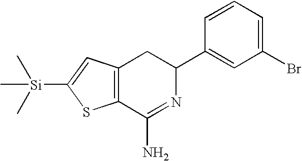

In the first reaction vessel, to a −10° C. stirred solution of 3-bromobenzaldehyde (0.12 mL, 1.02 mmol) in THF (2 mL) was added lithium bis(trimethylsilyl)amide (1.02 mL, 1.02 mmol) and the reaction was stirred at 0° C. for 2 hours. In the second reaction vessel, to a -78° C. stirred solution of freshly prepared LDA (0.11 g, 1.02 mmoles) in THF (2 mL) was slowly added DMPU (0.19 mL, 1.53 mmol) and Example 44 (0.20 g, 1.02 mmol) in THF (1 mL) and the anion stirred at −78° C. for 30 minutes. To this anion was quickly added the preformed silylimine via canula and the mixture stirred at −78° C. for 30 minutes. The mixture was warmed to 0° C. and stirred an additional 30 minutes. The reaction mixture was quenched with 1N HCl, extracted with CH₂Cl₂ (3×20 mL) and dried over Na₂SO₄. The solvent was removed under reduced pressure to yield the crude title compound as an amber oil. (0.39 g, quantitative). ¹H NMR (300 MHz, DMSO-d₆): δ 0.37 (s, 9H); 1.97 (m, 2H); 4.96 (m, 1H); 7.12 (s, 1H), 7.45 (m, 4H); 10.26 (br s, 1H); 10.75 (br s, 1H). HPLC (Platform 3): 2.34 minutes. m/z (APCI) 279 M, 281 M+2.

### EXAMPLE 46

### 5-(3-Bromophenyl)-4,5-dihydrothieno[2,3-c]pyridin-7-amine trifluoroacetate (Scheme #10, C)

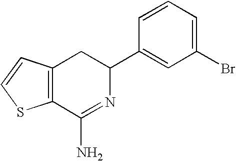

To a solution of crude Example 45 (0.39 g, 1.02 mmol) in THF (20 mL) was added tetrabutylammonium fluoride (1.50 mL, 1.53 mmol) and the mixture stirred at ambient temperature for 18 hours. The THF was removed under reduced pressure to yield an amber syrup. To this was added EtOAc (50 mL) and washed with sat. Na₂HCO₃ (2×25 n3L) and brine (1×25 mL). After drying over Na₂SO₄, the EtOAc was removed under reduced pressure to yield a yellow waxy solid. To this was added acetonitrile:water:TFA (75:25:0.1, 3 mL) and the resulting precipitate was removed. The filtrate was purified using RP-HPLC (Ret. time: 20.00 mins). The combined purified fractions were lyophilized to give the title compound as a white TFA salt (0.07 g, 40%). ¹H NMR (300 MHz, DMSO-d₆): δ 3.28 (br m, 2H); 5.09 (dd, J=8.4 Hz, 1H); 7.22 (d, J=4.8 Hz, 1H); 7.39 (m, 2H); 7.57 (m, 1H); 7.65 (s, 1H); 8.17 (d, J=4.8 Hz, 1H); 8.59 (br s, 1H); 9.50 (br s, 1H). HPLC (Platform 8): 1.58 minutes. m/z (APCI) 307 M, 309 M+2. Agilent preparative reverse phase HPLC conditions:

Compounds were purified on a Phenomenex Luna C18 reverse phase column (250×21 mm, 10 micron particle size). The crude compounds were solubilized in acteonitrile:water:TFA (75:25:0.1). An elution gradient (0% acetonitrile hold over 10 mins, 0-50% acteonitrile over 12 mins, hold at 50% acteonitrile for 3 mins, 50-100% acteonitrile over 7 mins, flow rate at 40 ml/min, 220 nm) yielded the purified title compounds.

### EXAMPLE 47

### 5-(3′-Methoxybiphenyl-3-yl)-4,5-dihydrothieno[2,3-c]pyridin-7-amine trifluoroacetate (Scheme #10, D)

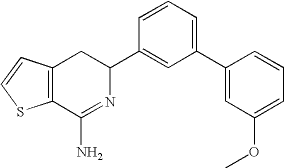

To a solution of Example 46 (0.007 g, 0.017 mmol) in 7:3:2 1,2-dimethoxyethane:water:ethanol (1 mL) was added tripotassium phosphate (0.009 g, 0.04 mmol), 3-methoxy-phenylboronic acid (0.005 g, 0.033 mmol), and dichlorobis(triphenylphosphine) palladium (II) (0.002 g, 0.002 mmol). The contents were sealed in a microwave reaction vessel and heated via microwave to 100° C. for 10 minutes. The solvent was removed under reduced pressure to yield a black oil. To this was added acetonitrile:water:TFA (75:25:0.1, 3 mL) and the resulting precipitate was removed. The filtrate was purified using RP-KPLC (Ret. time: 15.52 mins). The combined purified fractions were lyophilized to give the title compound as a white TFA salt (0.004 g, 57%).

¹H NMR (300 MFz, DMSO-d₆): δ 3.36 (br m, 2H); 3.83 (s, 3H); 5.16 (dd, J=6.6 Hz, 1H); 6.96 (d, J=7.8 Hz, 1H); 7.22 (m, 3H); 7.40 (m, 2H); 7.47 (t, J=7.8 Hz, 1H); 7.67 (d, J=7.8 Hz, 1H); 7.73 (s, 1H); 8.54 (d, J=4.8 Hz, 1H); 8.44 (br s, 1H); 9.45 (br s, 1H). HPLC (Platform 3): 2.06 minutes. m/z (APCI) 335 M+1.

Agilent preparative reverse phase HPLC conditions:

Compounds were purified on a Phenomenex Luna C18 reverse phase column (250×21 mm, 10 micron particle size). The crude compounds were solubilized in acteonitrile:water:TFA (75:25:0.1). An elution gradient (0-50% acteonitrile over 12 mins, hold at 50% acteonitrile for 3 mins, 50-100% acteonitrile over 7 mins, flow rate at 40 ml/min, 220 nm) yielded the purified title compounds.

Additional compounds are shown in Table 1.

TABLE 1

| | | | | m/z M + 1 | LC ₜ_{_{R}} |
|---|---|---|---|---|---|
| Example | Chemistry | Compound | NMR | Ionization | (min) |
| 48 | 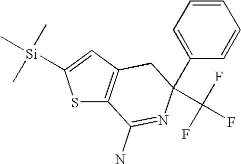 | 5-Phenyl-5-(trifluoromethyl)-2-(trimethylsilyl)-4,5-dihydrothieno[2,3-c]pyridin-7-aminetrifluoroacetate | ¹H NMR (300 MHz,DMSO-d6/TFA-d) δ0.32 (s, 9H), 3.74 (d, J =16.8 Hz, 1H), 4.19(d, J = 16.8 Hz, 1H),7.48 (m, 4H), 7.60 (m,2H), 9.02 (br s, 1H),10.18 (s, 1H) | 369(APCI+) | 2 |
| 49 | 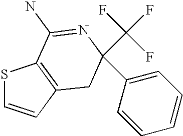 | 5-Phenyl-5-(trifluoromethyl)-4,5-dihydrothieno[2,3-c]pyridin-7-amine | ¹H NMR (300 MHz,DMSO-d6/TFA-d) δ3.73 (d, J = 17.1 Hz,1H), 4.22 (d, J = 17.1Hz, 1H), 7.27 (d, J =5.1 Hz, 1H), 7.48 (m,3H), 7.60 (m, 2H),8.18 (d, J = 5.1 Hz,1H), 8.99 (br s, 1H),10.19 (s, 1H) | 297(APCI+) | 1.65 |
| 50 | 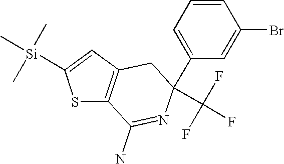 | 5-(3-Bromophenyl)-5-(trifluoromethyl)-2-(trimethylsilyl)-4,5-dihydrothieno[2,3-c]pyridin-7-aminetrifluoroacetate | ¹H NMR (300 MHz,DMSO-d6/TFA-d) δ0.34 (s, 9H), 3.75 (d, J =17.1 Hz, 1H), 4.23(d, J = 17.1 Hz, 1H),7.43 (m, 2H), 7.65 (m,2H), 7.82 (s, 1H), 9.03(br s, 1H), 10.23 (s,1H) | 447, 449(APCI+) | 2.20 |
| 51 | 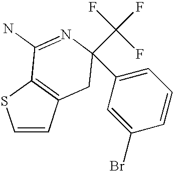 | 5-(3-Bromophenyl)-5-(trifluoromethyl)--4,5-dihydrothieno[2,3-c]pyridin-7-aminetrifluoroacetate | ¹H NMR (300 MHz,DMSO-d6/TFA-d) δ3.74 (d, J = 17.1 Hz,1H), 4.27 (d, J = 17.1Hz, 1H), 7.27 (d, J =5.1 Hz, 1H), 7.44 (t, J =8.1 Hz, 1H), 7.64 (m,2H), 7.83 (s, 1H), 8.20(d, J = 5.1 Hz, 1H),9.03 (br s, 1H),10.23 (s, 1H) | 375, 377(APCI+) | 1.84 |
| 52 | 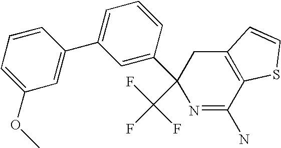 | 5-(3′-Methoxybiphenyl-3-yl)-5-(trifluoromethyl)-4,5-dihydrothieno[2,3-c]pyridin-7-aminetrifluoroacetate | ¹H NMR (300 MHz,DMSO-d6/TFA-d) δ3.78 (d, J = 17.4 Hz,1H), 3.84 (s, 3H), 4.37(d, J = 17.4 Hz, 1H),6.98 (d, J = 7.5 Hz,1H), 7.17 (s, 1H), 7.21(d, J = 7.5 Hz, 1H), 7.31 (d, J = 5.1 Hz,1H), 7.41 (t, J = 7.5Hz, 1H), 7.56 (m, 2H),7.73 (d, J = 7.5 Hz,1H), 7.85 (s, 1H), 8.19(d, J = 5.1 Hz, 1H),8.73 (br s, 1H), 10.16(s, 1H) | 403(APCI+) | 2.10 |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety.

1. A compound of formula I 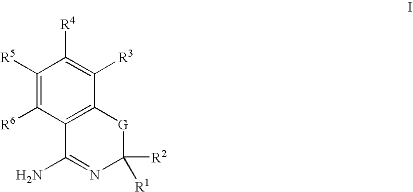
or a pharmaceutically acceptable salt, tautomer, or in vivo-hydrolysable precursor thereof, wherein:
G is O, NR⁷ or CR⁸R⁹;
R¹ is H, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 R¹⁴;
R² is Q or -L-Q;
or R¹ and R² together with the carbon atom to which they are attached form a 3-14 membered cycloalkyl group or 3-14 membered heterocycloalkyl group, each substituted by Cy² and optionally substituted by 1, 2, 3, 4 or 5 A⁴;
R³, R⁴, R⁵ and R⁶ are, independently, H, CN, NO₂, OR^{a}, SR^{a}, OC(O)R^{a}, OC(O)OR^{b}, OC(O)NR^{c}R^{d}, C(O)R^{a}, C(O)OR^{b}, C(O)NR^{c}R^{a}, NR^{c}R^{a}, NR^{c}C(O)R^{a}, NR^{c}C(O)OR^{d}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R⁷ is H, C(O)R^{a}, C(O)OR^{b}, C(O)NR^{c}R^{d}, S(O)R^{a}, S(O)₂R^{a}, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein the C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 R¹⁴;
R⁸ and R⁹ are, independently, H, CN, NO₂, OR^{a}, SR^{a}, OC(O)R^{a}, OC(O)OR^{b}, C(O)OR^{b}, OC(O)NR^{c}R^{d}, NR^{c}R^{d}, NR^{c}C(O)R^{a}, NR^{c}C(O)OR^{b}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
or R⁸ and R⁹ together with the carbon atom to which they are attached form a 3-14 membered cycloalkyl or 3-14 membered heterocycloalkyl group, each optionally substituted by 1, 2 or 3 R¹⁴;
R¹² and R¹³ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{b′}, S(O)₂R^{b′}, or S(O)₂NR^{c′}R^{d′};
R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
Q is aryl, cycloalkyl, heteroaryl or heterocycloalkyl, each optionally substituted by 1, 2, 3, 4 or 5 Cy¹ or A¹;
L is C₂₋₁₀ alkenylenyl, C₂₋₁₀ alkynylenyl, (CR¹²R¹³)_{q}, (CR¹²R¹³)_{q1}O(CR¹²R¹³)_{q2}, (CR¹²R¹³)_{q1}S(CR¹²R¹³)_{q2}, (CR¹²R¹³)_{q1}SO₂(CR¹²R¹³)_{q2}, (CR¹²R¹³)_{q1}SO(CR¹²R¹³)_{q2}, (CR¹²R¹³)_{q1}CO(CR¹²R¹³)_{q2}, (CR¹²R¹³)_{q1}NR^{e}(CR¹²R¹³)_{q2}, or (CR¹²R¹³)_{q1}CONR^{e}(CR¹²R¹³)_{q2};
Cy¹ is aryl, heteroaryl, cycloalkyl, or heterocycloalkyl, each optionally substituted with 1, 2, 3, 4 or 5 A²;
Cy² is aryl, heteroaryl, cycloalkyl, or heterocycloalkyl, each optionally substituted with 1, 2, 3, 4 or 5 A³;
A¹ is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, or S(O)₂NR^{c}R^{d};
A², A³, and A⁴ are each, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{e}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} is H, C₁₋₄ alkyl, C₁₋₄ haloalkyl, C₂₋₄ alkenyl, C₂₋₄ alkynyl, or CO—(C₁₋₄ alkyl);
q is 1, 2, 3, 4, 5 or 6;
q1 is 0, 1, 2 or 3; and
q2 is 0, 1, 2 or 3;
with the provisos:
a) when G is NH or CH₂; R² is -L-Q; L is —CH₂, —CH═CH—, or —C≡C—; and R¹ is H or methyl, then Q is other than unsubstituted phenyl; and
b) when G is NR⁷ or CR⁸R⁹; R⁷ is H, methyl, or phenyl optionally substituted by halo; R⁸ and R⁹ are each, independently, H or methyl; R² is Q; and R¹ is H or methyl, then Q is aryl, cycloalkyl, heteroaryl, or heterocycloalkyl, each substituted by at least one Cy³ and optionally substituted by 1, 2 or 3 A⁴.

2. A compound of claim 1 wherein R¹ is H, C₁₋₆ alkyl, C₁₋₆ haloalkyl, aryl, heteroaryl, arylalkyl, heteroarylalkyl, cycloalkylalkyl or heterocycloalkylalkyl, wherein the C₁₋₆ alkyl, C₁₋₆ haloalkyl, aryl, heteroaryl, arylalkyl, heteroarylalkyl, cycloalkylalkyl or heterocycloalkylalkyl is optionally substituted by 1, 2, 3, 4 or 5 R¹⁴.

3. A compound of claim 1 wherein R¹ is H, C₁₋₆ alkyl, C₁₋₆ haloalkyl, aryl, heteroaryl, arylalkyl or heteroarylalkyl, wherein the C₁₋₆ alkyl, aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

4. A compound of claim 1 wherein R¹ is C₁₋₆ haloalkyl, aryl, heteroaryl, arylalkyl or heteroarylalkyl, wherein the aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

5. A compound of claim 1 wherein:
R² is Q or -L-Q; and
Q is aryl, cycloalkyl, heteroaryl, or heterocycloalkyl, each optionally substituted by 1, 2 or 3 A¹.

6. A compound of claim 1 wherein:
R² is Q or -L-Q; and
Q is aryl, cycloalkyl, heteroaryl, or heterocycloalkyl, each substituted by at least one Cy¹ and optionally substituted by 1, 2 or 3 A¹.

7. A compound of claim 1 wherein:
R² is Q or -L-Q; and
Q is aryl or heteroaryl, each substituted by at least one Cy¹ and optionally substituted by 1, 2 or 3 A¹.

8. A compound of claim 1 wherein:
R² is Q or -L-Q; and
Q is aryl substituted by at least one Cy¹ and optionally substituted by 1, 2 or 3 A¹.

9. A compound of claim 1 wherein:
R² is Q or -L-Q; and
Q is phenyl substituted by at least one Cy¹ and optionally substituted by 1, 2 or 3 A¹.

10. A compound of claim 1 wherein:
R² is Q or -L-Q; and
Q is phenyl substituted by Cy¹.

11. A compound of claim 1 wherein:
R² is Q or -L-Q; and
Q is phenyl substituted by Cy¹; and
Cy¹ is aryl or heteroaryl, each optionally substituted with 1, 2, 3, 4 or 5 A².

12. A compound of claim 1 wherein:
R² is Q or -L-Q; and
Q is phenyl substituted by Cy¹; and
Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

13. A compound of claim 1 wherein:
R² is Q or -L-Q; and
Q is phenyl substituted by Cy¹, wherein the Cy¹ is substituted at the meta-position of the phenyl; and
Cy¹ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

14. A compound of claim 1 wherein R² is Q.

15. A compound of claim 1 wherein:
R² is -L-Q; and L is C₂₋₁₀ alkenylenyl, C₂₋₁₀ alkynylenyl or (CR¹²R¹³)_{q}.

16. A compound of claim 1 wherein:
R² is -L-Q; and L is (CR¹²R¹³)_{q}.

17. A compound of claim 1 wherein:
R² is -L-Q; and L is (CR¹²R¹³)_{q}; and q is 2.

18. A compound of claim 1 wherein:
R¹ and R² together with the carbon atom to which they are attached form a 3-14 membered cycloalkyl group or 3-14 membered heterocycloalkyl group, each substituted by Cy² and optionally substituted by 1, 2 or 3 A⁴;
Cy² is aryl or heteroaryl, each optionally substituted with 1, 2, 3, 4 or 5 A³.

19. A compound of claim 1 wherein:
R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
Cy² is aryl or heteroaryl, each optionally substituted with 1, 2 or 3 A³; and
A is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

20. A compound of claim 1 wherein:
R¹ and R² together with the carbon atom to which they are attached form a 3-14 membered cycloalkyl group substituted by Cy² and optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
Cy² is phenyl substituted with 1 or 2 A³; and
A³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

21. A compound of claim 1 wherein R³, R⁴, R⁵ and R⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴.

22. A compound of claim 1 wherein R³, R⁴, R⁵ and R⁶ are, independently, H, CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}. NR^{c′}C(O)R^{d′}, NR^{c′}C(O)OR^{a′} and NR^{c′}S(O)₂R^{b′}.

23. A compound of claim 1 wherein R³, R⁴, R⁵ and R⁶ are, independently, H.

24. A compound of claim 1 wherein R⁴ is CN, C(O)R^{a}, C(O)OR^{b}, C(O)NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, CN, NR^{c′}R^{d′}, NR^{c′}C(O)R^{d′}, NR^{c′}C(O)OR^{a′} and NR^{c′}S(O)₂R^{b′}.

25. A compound of claim 1 wherein G is O.

26. A compound of claim 1 wherein:
G is NR⁷ or CR⁸R⁹; and
R⁷, R⁸ and R⁹ are each, independently, H, C₁₋₁₀ alkyl, C₁₋₁₀ haloalkyl, C₂₋₁₀ alkenyl, C₂₋₁₀ alkynyl, cycloalkyl, heterocycloalkyl, cycloalkylalkyl or heterocycloalkylalkyl.

27. A compound of claim 1 wherein:
R¹ is C₁₋₆ haloalkyl, aryl, heteroaryl, arylalkyl or heteroarylalkyl, wherein the aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
R² is Q; and
Q is aryl or heteroaryl, each optionally substituted by 1, 2 or 3 A¹.

28. A compound of claim 1 wherein the compound has the structure of formula II: 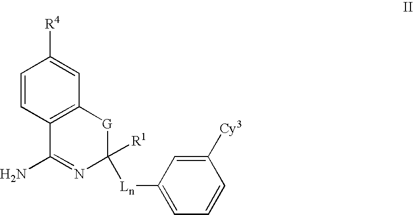
wherein:
R¹ is H, C₁₋₆ alkyl, C₁₋₆ haloalkyl, aryl, heteroaryl, arylalkyl or heteroarylalkyl, wherein the C₁₋₆ alkyl, aryl, heteroaryl, arylalkyl or heteroarylalkyl is optionally substituted by 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl;
L is C₁₋₄ alkylenyl;
n is 0 or 1;
Cy³ is aryl or heteroaryl, each optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

29. A compound of claim 28 wherein:
L is CH₂CH₂; and
Cy³ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

30. A compound of claim 1 wherein the compound has the structure of formula IIIa or formula IIIb: 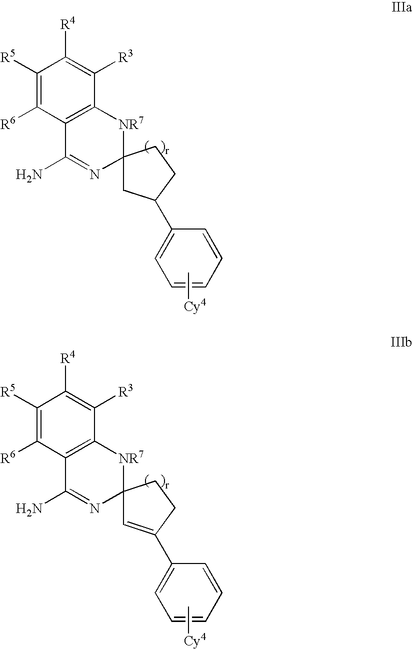
wherein:
r is 0, 1, 2 or 3; and
Cy⁴ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

31. A compound of claim 1 wherein the compound has the structure of formula IVa or formula IVb: 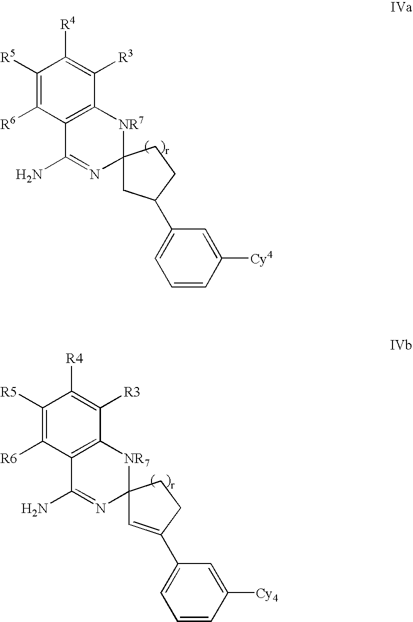
wherein:
r is 0, 1, 2 or 3; and
Cy⁴ is aryl optionally substituted with 1, 2 or 3 substituents independently selected from halo, CN, OH, C₁₋₆ alkoxy, C₁₋₆ haloalkoxy, C₁₋₆ haloalkyl, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl and heterocycloalkyl.

32. A compound of Formula V: 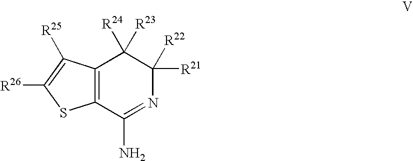
or a pharmaceutically acceptable salt, tautomer, or in vivo-hydrolysable precursor thereof, wherein:
R²¹ is H, C₁₋₆ alkyl, C₁₋₆ haloalkyl, aryl, heteroaryl, cycloalkyl, heterocycloalkyl, arylalkyl, heteroarylalkyl, cycloalkylalkyl or heterocycloalkylalkyl, wherein the C₁₋₆ alkyl, aryl, heteroaryl, cycloalkyl, heterocycloalkyl, arylalkyl, heteroarylalkyl, cycloalkylalkyl or heterocycloalkylalkyl is optionally substituted by 1, 2, 3, 4 or 5 R²⁹;
R²² is Q or -L-Q;
R²³, R²⁴, R²⁵ and R²⁶ are, independently, H, Si(C₁₋₁₀ alkyl)₃, CN, NO₂, OR^{a}, SR^{a}, OC(O)R^{a}, OC(O)OR^{b}, OC(O)NR^{c}R^{d}, C(O)R^{a}, C(O)OR^{b}, C(O)NR^{c}R^{d}, NR^{c}R^{d}, NR^{c}C(O)R^{a}, NR^{c}C(O)OR^{b}, NR^{c}S(O)₂R^{b}, S(O)R^{a}, S(O)NR^{c}R^{d}, S(O)₂R^{a}, S(O)₂NR^{c}R^{d}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R²⁹;
R²⁷ and R²⁸ are each, independently, H, halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b}, OC(O)NR^{c′}R^{d′}, NR^{c′}R^{d′}, NR^{c′}C(O)R^{d}, NR^{c′}C(O)OR^{a′}, NR^{c′}S(O)₂R^{b′}, S(O)R^{b′}, S(O)NR^{c′}R^{d′}, S(O)₂R^{b′}, or S(O)₂NR^{c′}R^{d′};
R²⁹ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b}, C(O)NR^{c′}R^{d′}, C(O)OR^{a}, OC(O)R^{b}, OC(O)NR^{c′}R^{d′}, NR^{c′}R^{d′}. NR^{c′}C(O)R^{d′}, NR^{c′}C(O)OR^{a′}, NR^{c′}S(O)₂R^{b′}, S(O)R^{b}, S(O)NR^{c′}R^{d′}, S(O)₂R^{b′}, or S(O)₂NR^{c′}R^{d′}; Q is aryl, cycloalkyl, heteroaryl or heterocycloalkyl, each optionally substituted by 1, 2, 3, 4 or 5 Cy¹ or A¹;
L is C₂₋₁₀ alkenylenyl, C₂₋₁₀ alkynylenyl, (CR²⁷R²⁸)_{q}, (CR²⁷R²⁸)_{q1}O(CR²⁷R²⁸)_{q2}, (CR²⁷R²⁸)_{q1}S(CR²⁷R²⁸)_{q2}, (CR²⁷R²⁸)_{q1}SO₂(CR²⁷R²⁸)_{q2}, (CR²⁷R²⁸)_{q1}SO(CR²⁷R²⁸)_{q2}, (CR²⁷R²⁸)_{q1}CO(CR²⁷R²⁸), (CR²⁷R²⁸)_{q1}, NR^{e}(CR²⁷R²⁸)_{q2}, or (C CR²⁷R²⁸)_{q1}CONR^{e}(C CR²⁷R²⁸)_{q2};
Cy¹ is aryl, heteroaryl, cycloalkyl, or heterocycloalkyl, each optionally substituted with 1, 2, 3, 4 or 5 A²;
A¹ is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{b}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl or heterocycloalkylalkyl, wherein each of the C₁₋₆ alkyl, C₂₋₆ alkenyl, C₂₋₆ alkynyl, arylalkyl, cycloalkylalkyl, heteroarylalkyl or heterocycloalkylalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{k}, NR^{c}S(O)₂₋₁e, S(O)le, S(O)NR^{c}R^{d}, S(O)₂R^{b}, or S(O)₂NR^{c}R^{d};
A² is halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{b}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of the C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2, 3, 4 or 5 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, NR^{c}S(O)R^{b}, NR^{c}S(O)₂R^{e}, S(O)R^{b}, S(O)NR^{c}R^{d}, S(O)₂R^{b}, or S(O)₂NR^{c}R^{d};
R^{a} and R^{a′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are each, independently, H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl, wherein the C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are each, independently, H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are each, independently, H, C₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein the C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} is H, C₁₋₄ alkyl, C₁₋₄ haloalkyl, C₂₋₄ alkenyl, C₂₋₄ alkynyl, or CO—(C₁₋₄ alkyl);
q is 1, 2, 3, 4, 5 or 6;
q1 is 0, 1, 2 or 3; and
q2 is 0, 1, 2 or 3;
with the provisos:
when R²¹, R²³ and R²⁴ are each H, and R²² is Q, then Q is aryl, cycloalkyl, heteroaryl, or heterocycloalkyl, each substituted by at least one Cy¹ and optionally substituted by 1, 2 or 3 A¹; and
when R²¹, R²³ and R²⁴ are each H, R²² is -L-Q and L is —C≡C—, then Q is other than unsubstituted phenyl.

33-69. (canceled)

70. A compound selected from:
3-(3′-Methoxybiphenyl-3-yl)-3,4-dihydroisoquinolin-1-amine trifluoroacetate;
3-(3-Bromophenyl)-3,4-dihydroisoquinolin-1-amine trifluoroacetate;
3-Biphenyl-3-yl-3,4-dihydroisoquinolin-1-amine trifluoroacetate;
3-Phenyl-3-(trifluoromethyl)-3,4-dihydroisoquinolin-1-amine trifluoroacetate;
3-(3′-Methoxybiphenyl-3-yl)-3-(trifluoromethyl)-3,4-dihydroisoquinolin-1-amine trifluoroacetate;
3-(3-Bromophenyl)-3-(trifluoromethyl)-3,4-dihydroisoquinolin-1-amine trifluoroacetate;
3-(3-Chlorophenyl)-3-phenyl-3,4-dihydroisoquinolin-1-amine trifluoroacetate;
3-(3′-Methoxybiphenyl-3-yl)-3-phenyl-3,4-dihydroisoquinolin-1-amine trifluoroacetate;
3-(3-Bromophenyl)-3-phenyl-3,4-dihydroisoquinolin-1-amine trifluoroacetate;
3-(3′-Methoxybiphenyl-3-yl)-3-methyl-3,4-dihydroisoquinolin-1-amine trifluoroacetate;
3-(3-Bromophenyl)-3-methyl-3,4-dihydroisoquinolin-1-amine;
3-(3-Bromophenyl)-1-(ethylthio)-3-methyl-3,4-dihydroisoquinoline;
3-Biphenyl-3-yl-3-methyl-3,4-dihydroisoquinolin-1-amine trifluoroacetate;
3-[2-(3′-Methoxybiphenyl-3-yl)ethyl]-3-methyl-3,4-dihydroisoquinolin-1-amine trifluoroacetate;
3-[2-(3-Bromophenyl)ethyl]-3-methyl-3,4-dihydroisoquinolin-1-amine trifluoroacetate;
3-[2-(3′-Methoxybiphenyl-3-yl)ethyl]-3-phenyl-3,4-dihydroisoquinolin-1-amine trifluoroacetate;
N-{[1-Amino-3-phenyl-3-(trifluoromethyl)-3,4-dihydroisoquinolin-6-yl]methyl}methanesulfonamide trifluoroacetate;
N-{[1-Amino-3-phenyl-3-(trifluoromethyl)-3,4-dihydroisoquinolin-6-yl]methyl}acetamide trifluoroacetate;
6-(Aminomethyl)-3-phenyl-3-(trifluoromethyl)-3,4-dihydroisoquinolin-1-amine bis trifluoroacetate;
3-Phenyl-6-(1H-tetrazol-5-yl)-3-(trifluoromethyl)-3,4-dihydroisoquinolin-1-amine trifluoroacetate;
1-Amino-3-phenyl-3-(trifluoromethyl)-3,4-dihydroisoquinoline-6-carboxylic acid trifluoroacetate;
1-Amino-3-phenyl-3-(trifluoromethyl)-3,4-dihydroisoquinoline-6-carbonitrile HCl salt;
1-Amino-3-(3′-methoxybiphenyl-3-yl)-3-(trifluoromethyl)-3,4-dihydroisoquinoline-6-carboxamide trifluoroacetate;
1-Amino-3-(3-bromophenyl)-3-(trifluoromethyl)-3,4-dihydroisoquinoline-6-carboxamide trifluoroacetate;
1-Amino-3-(3′-methoxybiphenyl-3-yl)-3-(trifluoromethyl)-3,4-dihydroisoquinoline-6-carboxylic acid trifluoroacetate;
1-Amino-3-(3′-methoxybiphenyl-3-yl)-3-(trifluoromethyl)-3,4-dihydroisoquinoline-6-carbonitrile trifluoroacetate;
1-Amino-3-(3-bromophenyl)-3-(trifluoromethyl)-3,4-dihydroisoquinoline-6-carbonitrile;
2-[2-(3′-methoxybiphenyl-3-yl)ethyl]-2-methyl-1,2-dihydroquinazolin-4-amine trifluoroacetate;
2-[2-(3-Bromophenyl)ethyl]-2-methyl-1,2-dihydroquinazolin-4-amine trifluoroacetate;
2-(3′-Methoxybiphenyl-3-yl)-2-methyl-1,2-dihydroquinazolin-4-amine trifluoroacetate;
2-(3-Bromophenyl)-2-methyl-1,2-dihydroquinazolin-4-amine trifluoroacetate;
4-Amino-2-[2-(3′-methoxybiphenyl-3-yl)ethyl]-2-methyl-1,2-dihydroquinazoline-7-carboxylic acid trifluoroacetate;
4-Amino-2-[2-(3-bromophenyl)ethyl]-2-methyl-1,2-dihydroquinazoline-7-carboxylic acid trifluoroacetate;
2-[2-(3′-Methoxybiphenyl-3-yl)ethyl]-1,2-dimethyl-1,2-dihydroquinazolin-4-amine trifluoroacetate;
2-[2-(3′-Methoxybiphenyl-3-yl)ethyl]-2-methyl-2H-1,3-benzoxazin-4-amine trifluoroacetate;
2-[2-(3-Bromophenyl)ethyl]-2-methyl-2H-1,3-benzoxazin-4-amine trifluoroacetate;
2-(3′-Methoxybiphenyl-3-yl)-2-methyl-2H-1,3-benzoxazin-4-amine trifluoroacetate;
2-(3-Bromophenyl)-2-methyl-2H-1,3-benzoxazin-4-amine;
2-(3-Bromophenyl)-N-methoxy-2-methyl-2H-1,3-benzoxazin-4-amine trifluoroacetate;
2-(3-Bromophenyl)-4-chloro-2-methyl-2H-1,3-benzoxazine;
2-(3-Bromophenyl)-2-methyl-2,3-dihydro-4H-1,3-benzoxazin-4-one;
3-(3′-Methoxybiphenyl-3-yl)-1′H-spiro[cyclohex-2-ene-1,2′-quinazolin]-4′-amine trifluoroacetate;
3-(3′-Methoxybiphenyl-3-yl)-1′H-spiro[cyclohexane-1,2′-quinazolin]-4′-amine trifluoroacetate;
3-Methyl-5-(trimethylsilyl)thiophene-2-carbonitrile;
5-(3-Bromophenyl)-2-(trimethylsilyl)-4,5-dihydrothieno[2,3-c]pyridin-7-amine;
5-(3-Bromophenyl)-4,5-dihydrothieno[2,3-c]pyridin-7-amine trifluoroacetate;
5-(3′-Methoxybiphenyl-3-yl)-4,5-dihydrothieno[2,3-c]pyridin-7-amine trifluoroacetate;
5-Phenyl-5-(trifluoromethyl)-2-(trimethylsilyl)-4,5-dihydrothieno[2,3-c]pyridin-7-amine trifluoroacetate;
5-Phenyl-5-(trifluoromethyl)-4,5-dihydrothieno[2,3-c]pyridin-7-amine;
5-(3-Bromophenyl)-5-(trifluoromethyl)-2-(trimethylsilyl)-4,5-dihydrothieno[2,3-c]pyridin-7-amine trifluoroacetate;
5-(3-Bromophenyl)-5-(trifluoromethyl)-4,5-dihydrothieno[2,3-c]pyridin-7-amine trifluoroacetate;
5-(3′-Methoxybiphenyl-3-yl)-5-(trifluoromethyl)-4,5-dihydrothieno[2,3-c]pyridin-7-amine trifluoroacetate
or a pharmaceutically acceptable salt, an alternative salt, tautomer, or in vivo-hydrolysable precursor thereof.

71. A pharmaceutical composition comprising as active ingredient a therapeutically effective amount of a compound according to claim 1 in association with a pharmaceutically acceptable excipient, carrier or diluent.

72-76. (canceled)

77. A method of inhibiting activity of BACE comprising contacting said BACE with a compound of claim 1.

78. A method of treating or preventing an Aβ-related pathology in a mammal, comprising administering to said patient a therapeutically effective amount of a compound of claim 1.

79. The method of claim 78, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

80. The method of claim 78, wherein said mammal is a human.

81. A method of treating or preventing an Aβ-related pathology in a mammal, comprising administering to said patient a therapeutically effective amount of a compound of claim 1 and at least one cognitive enhancing agent, memory enhancing agent, or choline esterase inhibitor.

82. The method of claim 81, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

83. The method of claim 81, wherein said mammal is a human.
